SEQUENCE LISTING (11 sequences)

SEQ ID NO: 1 - protein, 5 aa, synthetic construct
SYGMH

SEQ ID NO: 2 - protein, 17 aa, synthetic construct
IIWFDGSSTYYADSVRG

SEQ ID NO: 3 - protein, 9 aa, synthetic construct
ELGRRYFDL

SEQ ID NO: 4 - protein, 11 aa, synthetic construct
RASQSVSSYLA

SEQ ID NO: 5 - protein, 7 aa, synthetic construct
DASKRAT

SEQ ID NO: 6 - protein, 10 aa, synthetic construct
QQRSKWPPWT

SEQ ID NO: 7 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 8 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 9 - protein, 448 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 447 aa, synthetic construct
  MOD_RES 1: note = pyroglutamate residue
XVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0116985 A1
(43) Pub. Date: Apr. 30, 2026

(54) METHODS OF TREATING THYROID EYE DISEASE

(71) Applicant: Horizon Therapeutics Ireland DAC, Dublin (IE)

(72) Inventors: Jiayin HUANG, Buffalo Grove, IL (US); Majin CASTILLO, San Antonio, TX (US); Farah ALI, Wilmette, IL (US)

(73) Assignee: Horizon Therapeutics Ireland DAC, Dublin (IE)

(21) Appl. No.: 19/373,528

(22) Filed: Oct. 29, 2025

Related U.S. Application Data
(60) Provisional application No. 63/713,679, filed on Oct. 30, 2024.

(51) Int. Cl. C07K 16/28; A61K 9/00; A61K 39/00; A61K 47/10; A61K 47/20; A61K 47/22; A61K 47/26

(52) U.S. Cl. CPC C07K 16/2863; A61K 9/0019; A61K 47/10; A61K 47/20; A61K 47/22; A61K 47/26; A61K 2039/505; A61K 2039/54; A61K 2039/545; C07K 2317/565; C07K 2317/76

(57) ABSTRACT

The present invention relates to methods for treating or reducing the severity of thyroid eye disease. In particular, the present invention provides methods for treating or reducing the severity of thyroid eye disease in patients in need thereof comprising administering an IGF-1R antagonist antibody according to specific dosage regimens. Pharmaceutical compositions comprising the IGF-1R antagonist antibody for use in the methods are also described.

### CROSS-REFERENCE TO RELATED APPLICATIONS

This application claims the benefit of U.S. Provisional Application No. 63/713,679, filed Oct. 30, 2024, which is hereby incorporated by reference in its entirety.

### DESCRIPTION OF THE TEXT FILE SUBMITTED ELECTRONICALLY

The present application contains a Sequence Listing, which has been submitted electronically in XML format and is hereby incorporated by reference in its entirety. The computer readable format copy of the Sequence Listing, which was created on Oct. 29, 2025, is named 11023-US02-SEC_ST26.xml and is 12,273 bytes in size.

### FIELD OF THE INVENTION

The present invention relates to the fields of ophthalmology, endocrinology, and biopharmaceuticals. In particular, the present invention relates to methods for the treatment of thyroid eye disease by administering an antibody that inhibits the human insulin-like growth factor 1 receptor (IGF-1R) according to specific dosage regimens.

### BACKGROUND OF THE INVENTION

Thyroid eye disease (TED), also termed Graves' ophthalmopathy/orbitopathy and thyroid-associated ophthalmopathy, is a serious, debilitating and painful autoimmune disease that can, in severe cases, lead to blindness. TED is commonly associated with Graves' hyperthyroidism/disease, but also occurs in a proportion of patients with other autoimmune thyroid diseases, including Hashimoto's thyroiditis. The natural history involves “active TED,” which is an autoimmune inflammatory response targeting orbital soft tissues and “inactive TED,” in which there is tissue expansion remodeling. Active TED typically lasts 1 to 3 years, and then the inflammation spontaneously subsides to leave the pathology of inactive TED (Burch and Wartofsky, Endocrine Reviews, Vol. 14(6): 747-793, 1993).

The annual incidence rate of TED in the US has been estimated to be 16 cases per 100,000 people for women and 2.9 cases per 100,000 people for men (Bartley, Trans Am Ophthalmol Soc., Vol. 92: 477-588, 1994). The incidence appears to be comparable in Europe (Abraham-Nordling et al., Eur J Endocrinol., Vol. 165(6): 899-905, 2011; Mostbeck et al., Eur J Nucl Med., Vol. 25(4):367-374, 1998; Noth et al., Swiss Med Wkly., Vol. 131(41-42):603-609, 2001; Tanda et al., J Clin Endocrinol Metab., Vol. 98(4):1443-1449, 2013). Patients aged between 30 and 50 years are most frequently affected, with severe cases more frequent in those older than 50 years (Dickinson, Clinical manifestations. In WWM Wiersinga & GJ Kahaly (Eds.). Graves' orbitopathy: a multidisciplinary approach—questions and answers (3rd ed.), 2017). The occurrence and severity of TED are associated with smoking (Prummel and Wiersinga, JAMA, Vol. 269(4):479-482, 1993).

A mounting body of evidence in the scientific literature indicates that the pathophysiology of active TED involves autoimmune activation and proliferation of orbital fibroblasts (Bahn, N Engl J Med., Vol. 362(8):726-738, 2010; Boschi et al., Br J Ophthalmol., Vol. 89(6):724-729, 2005; Smith, Pharmacol Rev., Vol. 62(2):199-236, 2010). The activation of fibroblasts triggers release of inflammatory cytokines, infiltration of immune cells into orbital soft tissues (muscle, interstitial and adipose), excessive synthesis of extracellular matrix, and tissue expansion and fibrotic remodeling. During the inactive phase, inflammation is absent and the disease plateaus, but significant remodeling of orbital tissue remains and rarely does the patient return to baseline.

Clinical features of TED include orbital pain, swelling, dry eye, redness and discomfort of the lids and ocular surface, thickening and retraction of the eyelids and proptosis (exophthalmos) due to the expansion of tissue behind the eye (Bahn, 2010; Burch and Wartofsky, 1993; Dickinson, 2017; Mallika et al., Malays Family Physician, Vol. 4(1):8-14, 2009). Although TED is heterogenous and variable in presentation, proptosis is one of the most prevalent and widely known symptoms of TED. TED has high morbidity (Bartalena et al., European Journal of Endocrinology, Vol. 158(3):273-285, 2008; Bartley, 1994; Dickinson, 2017; Gerding et al., Thyroid, Vol. 7(6):885-889, 1997), which takes the form of orbital pain, together with a number of serious, vision- or sight-threatening conditions, including diplopia (due to inability to correctly align the eyes), corneal ulceration (due to inability to close lids) and dysthyroid optic neuropathy (due to proptosis, tissue crowding and stress on the optic nerve). These combine to produce marked reductions in quality of life (e.g., physical functioning, role functioning, social functioning, mental health, health perceptions and pain) (Gerding et al, 1997; Terwee et al., Eur J Endocrinol., Vol. 146(6):751-757, 2002). TED can also produce profound psychosocial problems, in particular anxiety and depression, due to the alarming and disfiguring changes in appearance (Bartley et al., Ophthalmology, Vol. 103(6): 958-962, 1996; Coulter et al., Eur J Endocrinol., Vol. 157(2):127-131, 2007; Kahaly and Petrak et al., Clin Endocrinol (Oxf)., Vol. 63(4):395-402, 2005). Taken together, these data show that TED is a physically and emotionally debilitating condition.

Teprotumumab is a fully human immunoglobulin G1 monoclonal antibody directed against human insulin-like growth factor-1 receptor (IGF-1R). The IGF-1R is a tyrosine kinase cell surface receptor that shares ˜50% overall homology with the insulin receptor (Ullrich et al., EMBO J., Vol. 5(10):2503-2512, 1986). Teprotumumab binds with high affinity and selectivity to the extracellular domain of IGF-1R and prevents its activation by the endogenous ligands, IGF-1 and IGF-2. Teprotumumab has no partial agonist activity at IGF-1R, as assessed by activation of the canonical signaling pathway (phosphoinositide 3 kinase/Akt) and has no affinity for the insulin receptor. In addition, teprotumumab causes direct inactivation of IGF-1R through antibody-induced cellular internalization and degradation. Binding of teprotumumab has been shown to inhibit canonical signal transduction and cellular proliferation and survival functions mediated by IGF-1R in cancer cells. Teprotumumab does not induce antibody-dependent cellular cytotoxicity.

Clinical trials of intravenously administered teprotumumab for the treatment of TED include three independent, randomized, double-masked, placebo-controlled, parallel-group, multicenter trials: Phase 2 Trial TED01RV (Smith et al., N Engl J Med., Vol. 376(18):1748-1761, 2017); Phase 3 Trial HZNP-TEP-301 (OPTIC) (Douglas et al., N Engl J Med., Vol. 382(4):341-352, 2020), and Phase 4 HZNP-TEP-403 Trial (Douglas et al., The Journal of Clinical Endocrinology & Metabolism, Vol. 109 (1): 25-35, 2024), and an open-label extension of Trial HZNP-TEP-301 (Trial HZNP-TEP-302; OPTIC-λ; Douglas et al., Ophthalmology, Vol. 129:438-449, 2022). In the phase 2 and phase 3 randomized, double-masked trials in patients with acute TED, teprotumumab resulted in statistically significant and clinically relevant improvements in measures that assessed multiple facets of TED (proptosis, inflammation as measured by Clinical Activity Score (CAS), diplopia and quality of life). In addition, the persistence of effect was demonstrated after approximately 1 year off treatment. In the phase 4 trial in which patients with inactive TED were enrolled, teprotumumab produced a statistically significant reduction in proptosis as compared to placebo and also resulted in reductions in orbital fat and extraocular muscle volumes as measured by magnetic resonance imaging and improvements in quality of life. Teprotumumab was approved in the United States for the treatment of thyroid eye disease in January 2020. Intravenously administered teprotumumab has a well-established and well-described safety profile in adults based on clinical trial participant exposure and post-marketing experience.

Although the approved intravenous dosing regimen of teprotumumab effectively treats TED, there is a need in the art to develop alternative effective dosing regimens with less invasive routes of administration to provide treatment options for those patients who are not able to tolerate intravenous infusions.

### SUMMARY OF THE INVENTION

The present invention is based, in part, on the identification of subcutaneous dosing regimens of an IGF-1R antagonist antibody, particularly teprotumumab, for effectively treating or reducing the severity of TED in a patient in need thereof. Accordingly, in certain embodiments, the present invention provides methods of treating TED in a patient in need thereof comprising administering subcutaneously to the patient an IGF-1R antagonist antibody described herein at a fixed dose once every two weeks. The fixed dose of the IGF-1R antagonist antibody can be from about 1,500 mg to about 2,000 mg, such as from about 1,500 mg to about 1,650 mg, 1,675 mg to about 1,850 mg, or from about 1,575 mg to about 1,750 mg. In some embodiments of the methods of the invention, the IGF-1R antagonist antibody is administered to the patient at a fixed dose of about 1,575 mg to about 1,750 mg once every two weeks. In one embodiment, the IGF-1R antagonist antibody is administered to the patient at a fixed dose of about 1,575 mg once every two weeks. In another embodiment, the IGF-1R antagonist antibody is administered to the patient at a fixed dose of about 1,750 mg once every two weeks.

Patients to be treated according to the methods of the invention may, in some embodiments, have or be diagnosed with moderate to severe TED. In such embodiments, the patient may have one or more of the following: lid retraction ≥2 mm, moderate or severe soft tissue involvement, proptosis ≥3 mm above normal for race and gender, and inconstant or constant diplopia (Gorman score 2-3). In some such embodiments, the patient has diplopia (intermittent, inconstant, or constant diplopia; Gorman score 1-3) prior to administration of the IGF-1R antagonist antibody. In further embodiments, the patient has an increase in proptosis of 3 mm or more in at least one eye prior to administration of the IGF-1R antagonist antibody.

In some embodiments, patients to be treated according to the methods of the invention may have or be diagnosed with active TED. In some such embodiments, the patient may have a clinical activity score (CAS) of 3 or more on the 7-component scale or a CAS of 4 or more on the 10-component scale in at least one eye. In other embodiments, patients to be treated according to the methods of the invention may have or be diagnosed with inactive TED. In such embodiments, the patient may have a CAS no more than 2 on the 7-component scale or 3 on the 10-component scale in either eye. In certain embodiments, a patient with inactive TED may have diplopia, an increase in proptosis, or restricted eye motility in any direction of gaze.

In particular embodiments, patients to be treated according to the methods of the invention have not previously had orbital irradiation, orbital decompression surgery, or strabismus surgery. Administration of the IGF-1R antagonist antibody according to the methods of the invention preferably delays or eliminates the need for these procedures.

In some embodiments of the methods of the invention, a patient is administered the IGF-1R antagonist antibody over a set treatment period, such as over the course of 12 weeks, 18 weeks, 24 weeks, 36 weeks, or 48 weeks. In certain embodiments, a patient is administered the IGF-1R antagonist antibody over the course of 24 weeks. In these and other embodiments, proptosis is reduced in at least one eye of the patient (e.g. by at least 2 mm) as compared to the patient's pre-treatment baseline following administration of the IGF-1R antagonist antibody. Administration of the IGF-1R antagonist antibody according to the methods of the invention can also reduce the severity of diplopia (e.g. by at least 1 grade), a patient's CAS (e.g. by at least two points), extraocular muscle volume (e.g. by at least 25%), and orbital fat volume (e.g. by at least 30%) as compared to the patient's pre-treatment baseline measurements. In certain embodiments, administration of the IGF-1R antagonist antibody according to the methods of the invention completely resolves diplopia in the patient (i.e. the patient has a Gorman score 0 following administration of the IGF-1R antagonist antibody). In certain other embodiments, administration of the IGF-1R antagonist antibody according to the methods of the invention reduces a patient's CAS to 0 or 1 following administration of the IGF-1R antagonist antibody.

The present invention also provides methods of improving one or more aspects of the quality of life of a patient with TED. For instance, administration of the IGF-1R antagonist antibody according to the methods of the invention improves visual function and/or visual appearance in the patient as measured by the Graves' Ophthalmopathy Quality of Life (GO-QoL) questionnaire. In some embodiments, administration of the IGF-1R antagonist results in an increase of 8 or more points on the visual function subscale and/or the visual appearance subscale of the GO-QoL.

The IGF-1R antagonist antibodies for use in the methods of the invention specifically bind to human IGF-1R and inhibit its activation and downstream signaling. In some embodiments, the IGF-1R antagonist antibody is a human monoclonal antibody of the IgG type. In one embodiment, the IGF-1R antagonist antibody is an IgG1 antibody. In certain embodiments, the IGF-1R antagonist antibody administered to a patient according to the methods of the invention comprises (i) a heavy chain variable region comprising a CDRH1 having the sequence of SEQ ID NO: 1, a CDRH2 having the sequence of SEQ ID NO: 2, and a CDRH3 having the sequence of SEQ ID NO: 3, and (ii) a light chain variable region comprising a CDRL1 having the sequence of SEQ ID NO: 4, a CDRL2 having the sequence of SEQ ID NO: 5, and a CDRL3 having the sequence of SEQ ID NO: 6. In related embodiments, the IGF-1R antagonist antibody comprises a heavy chain variable region comprising the sequence of SEQ ID NO: 7, and a light chain variable region comprising the sequence of SEQ ID NO: 8. In some embodiments, the IGF-1R antagonist antibody administered to a patient according to the methods of the invention comprises a heavy chain comprising the sequence of SEQ ID NO: 9, and a light chain comprising the sequence of SEQ ID NO: 10. In a preferred embodiment, the IGF-1R antagonist antibody administered to a patient according to the methods of the invention is teprotumumab.

The present invention also provides pharmaceutical compositions comprising an IGF-1R antagonist antibody, such as teprotumumab, for use in the methods of the invention described herein. The pharmaceutical compositions can comprise one or more pharmaceutically acceptable excipients. In certain embodiments, the pharmaceutical compositions comprise an IGF-1R antagonist antibody at a concentration from about 150 mg/mL to about 200 mg/mL, a buffer, one or more stabilizers, and a surfactant. For instance, in some embodiments, the pharmaceutical composition comprises about 150 mg/mL to about 200 mg/mL of the IGF-1R antagonist antibody, about 15 mM to about 25 mM histidine, about 200 mM to about 275 mM trehalose, and about 0.005% (w/v) to about 0.05% (w/v) polysorbate 20, wherein the composition has a pH of about 5.0 to about 6.0. In one such embodiment, the pharmaceutical composition comprises about 150 mg/mL of the IGF-1R antagonist antibody, about 20 mM histidine, about 250 mM trehalose, and about 0.01% (w/v) polysorbate 20, wherein the composition has a pH of about 5.5. In other embodiments, the pharmaceutical composition comprises about 150 mg/mL to about 200 mg/mL of the IGF-1R antagonist antibody, about 15 mM to about 25 mM histidine, about 200 mM to about 275 mM trehalose, about 30 mM to about 50 mM methionine, and about 0.05% (w/v) to about 0.35% (w/v) poloxamer 188, wherein the composition has a pH of about 5.0 to about 6.0. In one particular embodiment, the pharmaceutical composition comprises about 150 mg/mL of the IGF-1R antagonist antibody, about 20 mM histidine, about 210 mM trehalose, about 40 mM methionine, and about 0.2% (w/v) poloxamer 188, wherein the composition has a pH of about 5.5. Any of the pharmaceutical compositions described herein can be incorporated into injection devices, such as pre-filled syringes, autoinjectors, injection pumps, on-body injectors, and patch injectors for subcutaneous administration to a patient according to the methods described herein. In some embodiments, the IGF-1R antagonist antibody (e.g. teprotumumab) or pharmaceutical composition comprising the IGF-1R antagonist antibody (e.g. teprotumumab) is administered to the patient with an on-body injection device.

The use of IGF-1R antagonist antibodies in any of the methods disclosed herein or for preparation of medicaments for administration according to any of the methods disclosed herein is specifically contemplated. For instance, the present invention includes an IGF-1R antagonist antibody for use in a method of treating TED in a patient in need thereof, wherein the method comprises administering subcutaneously to the patient the IGF-1R antagonist antibody at any of the fixed doses described herein once every two weeks. The present invention also encompasses the use of an IGF-1R antagonist antibody for preparation of a medicament for treating TED in a patient in need thereof, wherein the medicament is administered or formulated for administration at a subcutaneous fixed dose as described herein once every two weeks.

### BRIEF DESCRIPTION OF THE DRAWINGS

FIGS. 1A-1B depict the simulated time-course PK profiles for TED patients receiving a fixed dose teprotumumab subcutaneous (SC) once every 2 weeks (Q2W) regimen with the high concentration formulation (HCF) at a dose of 1,575 mg (FIG. 1A) or 1,732.5 mg (FIG. 1B) as compared to patients receiving the intravenous (IV) once every 3 weeks (Q3W) body weight dosing regimen with the lyophilized formulation (lyo). The top line in each of the figures is the median value for the IV regimen, whereas the bottom line in each of the figures is the median value for the SC regimen. The shading represents the 5^{th} to 95^{th} percentiles for each dosing regimen.

FIGS. 2A-2B show a comparison of exposure metrics (Cmin,ss; Cavg,ss; and Cmax,ss) between a fixed dose teprotumumab SC Q2W regimen with the high concentration formulation (HCF) at a dose of 1,575 mg (FIG. 2A) or 1,732.5 mg (FIG. 2B) and an IV Q3W body weight dosing regimen with the lyophilized formulation (lyo). Points are the simulated exposure metrics. The boxes represent the 25^{th} to 75^{th} percentiles (the lower and upper quartiles, respectively). The horizontal line in the middle of each box represents the median. The whiskers represent the range of data points within 1.5 times the interquartile range.

### DETAILED DESCRIPTION

Teprotumumab, when administered intravenously, has been shown to result in many clinical benefits for patients with TED of varying levels of severity and disease duration. Not only has therapy with teprotumumab improved proptosis (eye bulging) comparable to the levels achieved with orbital decompression surgery but has also significantly improved disease activity and diplopia (double vision) resulting in clinically meaningful improvements in patients' quality of life. Non-intravenous dosing regimens that provide comparable (or even improved) efficacy and safety profiles would provide patients with treatment options that are less invasive, easier to administer, and available for patients who are not willing or able to receive intravenous infusions. The present invention addresses this need by providing novel subcutaneous fixed dosing regimens of an IGF-1R antagonist antibody (e.g. teprotumumab) for the treatment of TED. Accordingly, in certain embodiments, the present invention provides methods of treating TED in a patient in need thereof comprising administering subcutaneously an effective amount of an IGF-1R antagonist antibody according to specific dosage regimens described herein. The present invention also includes an IGF-1R antagonist antibody for use in a method of treating TED in a patient in need thereof, wherein the method comprises administering subcutaneously an effective amount of the IGF-1R antagonist antibody according to specific dosage regimens described herein. In some embodiments, the present invention provides a use of an IGF-1R antagonist antibody for preparation of a medicament for treating TED in a patient in need thereof, wherein the medicament is formulated for administration subcutaneously according to any of the dosage regimens described herein.

The term “treatment” or “treat” as used herein refers to the application or administration of the IGF-1R antagonist antibody to a patient who has or is diagnosed with TED, has one or more symptoms of TED, is at risk of developing TED, or had a predisposition to TED for the purpose of healing, alleviating, relieving, altering, ameliorating, or improving TED, one or more symptoms of TED, the risk of developing TED, or predisposition toward TED. The term “treatment” encompasses any improvement of the disease in the patient, including the slowing or halting of the progression of TED in the patient, a decrease in the number or severity of symptoms of TED, or an increase in frequency or duration of periods where the patient is free from the symptoms of TED. The term “patient,” used interchangeably herein with “subject” or “individual,” refers to individuals diagnosed with, suspected of being afflicted with, or at-risk of developing at least one disease (e.g. TED) for which the described compositions and methods are useful for treating. In certain embodiments, the patient is a human patient.

Thyroid eye disease (TED), also known as thyroid-associated ophthalmopathy (TAO), Graves' ophthalmopathy, or Graves' orbitopathy, is an autoimmune disease that is often progressive and affects the eye muscles and fatty tissue behind the eyes. The muscles and fatty tissues become inflamed leading to proliferation of cells and expansion of the orbital fat tissue and extraocular muscles causing proptosis (eye bulging), diplopia (double vision), orbital pain, and in severe cases, loss of vision due to compression of the optic nerve. TED is often associated with thyroid disorders, such as hyperthyroidism caused by Graves' disease, but can also develop with other thyroid diseases, such as Hashimoto's thyroiditis or even in individuals with normal thyroid levels. Symptoms of TED include swelling of the eyes and eyelids, dry or watery eyes, gritty feeling in the eyes, redness of the eyes and eyelids, pain in or behind the eyes, particularly with eye movement, difficulty closing eyes (retraction of the lids), corneal ulcerations, proptosis, and diplopia. TED is a heterogeneous disease that can be characterized by acute and chronic phases with varying levels of disease activity and duration.

In certain embodiments, a patient to be treated according to the methods of the invention has or is diagnosed with active TED. Active TED generally refers to phases of the disease characterized by inflammation and tissue damage. In some embodiments, active TED can be diagnosed using the Clinical Activity Score (CAS). The CAS is a tool that typically consists of the following seven components:
1. spontaneous retrobulbar pain,
2. pain on attempted eye movements (upward, side-to-side, and downward gazes),
3. redness of the eyelids,
4. redness of the conjunctiva,
5. swelling of the eyelids,
6. inflammation of the caruncle and/or plica, and
7. conjunctival swelling/edema (also known as chemosis).
   Each component is scored as present (1 point) or absent (0 points) for each eye or the most severely affected eye. The score at each assessment is the sum of all items present giving a range of 0-7. See Burch et al., Eur Thyroid J., Vol. 11(6):e220189, 2022. A ten-item CAS is also sometimes used, which includes the following additional three components:
8. increase of at least 2 mm in proptosis,
9. decrease of at least 8° in any duction, and
10. decrease of visual acuity by two lines on the Snellen chart.
   A CAS of ≥3/7 on the 7-component scale and a CAS of ≥4/10 on the 10-component scale is indicative of active TED. Active TED may also be diagnosed if the patient has a history or documentation of progression of TED based on subjective or objective worsening of vision, soft tissue inflammation, motility, or proptosis independently of the CAS. In some embodiments, the patient with active TED to be treated according to the methods of the invention has a CAS of ≥3 on the 7-component scale (i.e. a CAS of 3 to 7) in at least one eye. In other embodiments, the patient to be treated with active TED according to the methods of the invention has a CAS of ≥4 on the 10-component scale (i.e. a CAS of 4 to 10) in at least one eye.

In other embodiments, a patient to be treated according to the methods of the invention has or is diagnosed with inactive TED. Inactive TED refers to the phases of the disease where the inflammatory aspects are less pronounced, but the patient is still experiencing symptoms affecting their quality of life, including proptosis and/or diplopia, arising from the continued tissue expansion and fibrosis behind the eyes. Inactive TED can be diagnosed using the CAS (either the 7-component or 10-component scale). A CAS of ≤2/7 on the on the 7-component scale and a CAS of ≤3/10 on the 10-component scale can be used to diagnose inactive TED in a patient. In certain embodiments, the patient with inactive TED to be treated according to the methods of the invention has a CAS of ≤2 on the 7-component scale (i.e. a CAS of 0 to 2) in both eyes. In other embodiments, the patient with inactive TED to be treated according to the methods of the invention has a CAS of ≤3 on the 10-component scale (i.e. a CAS of 0 to 3) in both eyes. In yet other embodiments, the patient with inactive TED to be treated according to the methods of the invention has a CAS of 0 or 1 in at least one eye on either the 7-component or 10-component scale. In still other embodiments, the patient with inactive TED to be treated according to the methods of the invention has a CAS of 0 or 1 in both eyes on either the 7-component or 10-component scale.

TED has also been categorized into acute or chronic TED based on duration of the disease. Acute TED has been considered to be a disease duration of less than about 12 months, whereas chronic TED is typically considered to have a disease duration greater than 12 months. The understanding of the natural history of TED has evolved in recent years and evidence indicates that TED should be viewed as a progressive, heterogenous, autoimmune disease with a patient experiencing both active and inactive disease periods throughout the course of the disease. For instance, some patients that may have chronic and inactive disease can experience a resurgence of active disease (e.g. a flare) at some point in their disease course.

Severity of TED is classified into three different categories according to various clinical guidelines (see, e.g., Burch et al., Eur Thyroid J., Vol. 11(6):e220189, 2022; and Bartalena et al., Eur J Endocrinol., Vol. 185(4):G43-G67, 2021): (i) mild TED, (ii) moderate to severe TED, and (iii) sight-threatening TED. Mild TED is diagnosed in patients whose features of thyroid eye disease have only a minor impact on daily life insufficient to justify immunosuppressive or surgical treatment. Patients with mild TED usually have only one or more of the following: minor lid retraction (<2 mm), mild soft tissue involvement, proptosis <3 mm above normal for race and gender, transient or no diplopia, and corneal exposure responsive to lubricants. Moderate to severe TED is diagnosed in patients without sight-threatening disease whose thyroid eye disease has sufficient impact on daily life to justify the risks of medical or surgical intervention. Patients with moderate to severe thyroid eye disease usually have any one or more of the following: lid retraction ≥2 mm, moderate or severe soft tissue involvement, proptosis ≥3 mm above normal for race and gender, and inconstant or constant diplopia (Gorman score 2-3). Sight-threatening TED is diagnosed in patients with dysthyroid optic neuropathy (DON) and/or corneal breakdown, and/or globe subluxation (globe dislocation). In certain embodiments, a patient to be treated according to the methods of the invention is diagnosed with moderate to severe TED.

Classification of the activity and severity of TED also takes into account the impact of TED symptoms on a patient's quality of life. Quality of life of a TED patient can be assessed by the Graves' ophthalmopathy quality of life (GO-QoL) questionnaire (see Terwee et al., Br. J. Ophthalmol. Vol. 82(7):773-779, 1998). The GO-QoL is a 16-item self-administered questionnaire divided into two subscales that is used to measure changes over time in visual functioning and appearance of a TED patient. The first subscale (visual function subscale) relates to the impact of visual function on daily activities, whereas the second subscale (visual appearance subscale) relates to the impact of self-perceived appearance. The visual function subscale has 8 questions which are answered with one of the three following choices: (i) Yes—seriously limited, (ii) yes—a little limited, or (iii) no—not at all limited. The appearance subscale also has 8 questions which are answered with one of the three following choices: (i) Yes—very much so; (ii) Yes—a little; or (iii) No—not at all. Each question is scored 0-2, respectively, and the total raw score is then mathematically transformed to a 0-100 scale, where 0 represents the most negative impact on quality of life, and 100 represents no impact. A change of ≥8 points on the 0-100 scale is considered to be clinically meaningful. The combined score takes raw scores from both subscales and again transforms them to a single 0-100 scale.

In some embodiments, a patient to be treated according to the methods of the invention has diplopia prior to administration of the IGF-1R antagonist antibody (i.e. at baseline). Severity of diplopia can be assessed using the Gorman score according to the following scale:
0=no diplopia;
1=intermittent (diplopia in primary position of gaze, when tired or when first awakening);
2=inconstant (diplopia at extremes of gaze);
3=constant (continuous diplopia in primary or reading position)
   In one embodiment, the patient has intermittent diplopia prior to administration of the IGF-1R antagonist antibody. In another embodiment, the patient has inconstant diplopia prior to administration of the IGF-1R antagonist antibody. In yet another embodiment, the patient has constant diplopia prior to administration of the IGF-1R antagonist antibody. In still another embodiment, the patient has inconstant or constant diplopia prior to administration of the IGF-1R antagonist antibody.

In certain embodiments, a patient to be treated according to the methods of the invention has an increase in proptosis (also referred to as exophthalmos) of 3 mm or more in at least one eye prior to administration of the IGF-1R antagonist antibody (i.e. at baseline). The increase in proptosis can be relative to the patient's prior measurements (e.g. prior to diagnosis of TED) or relative to the normal average for the patient's race and gender. Proptosis refers to the forward projection, displacement, bulging, or protrusion of the globe anteriorly out of the orbit. Although it is generally accepted that the normal range of proptosis is 12-21 mm, it must be noted that the value for a normal person varies by age, gender and race. For example, in normal adult white males, the average distance of globe protrusion is 16.5 mm, with the upper limit of normal at 21.7 mm. In adult African Americans it averages 18.2 mm, with an upper normal limit of 24.1 mm in males and 22.7 mm in females. In Mexican adults, males averaged 15.2 mm and females averaged 14.8 mm and in Iran, for the age group of 20-70 years, the average was 14.7 mm. In Taiwanese adults, comparing normal subjects to those with Graves' Ophthalmopathy, the normal group had an average measurement of 13.9 mm versus 18.3 mm for the TAO group. Even within a group of people, there can be variability. Four ethnic groups in Southern Thailand had proptosis measurement averages ranging from 15.4 mm to 16.6 mm. In 2,477 Turkish patients, the median measurement was 13 mm, with an upper limit of 17 mm; and in a Dutch study, the upper limit was 20 mm in males and 16 mm in females. Although the average and upper limits for proptosis vary widely, it is accepted in the field that a difference greater than 2 mm between the eyes is significant and not normal. In certain embodiments, a patient to be treated according to the methods of the invention has a proptosis measurement of at least 18 mm in at least one eye prior to administration of the IGF-1R antagonist antibody (i.e. at baseline).

Measurement of the degree of proptosis can be performed using an exophthalmometer (e.g. Hertel exophthalmometer), which is an instrument used for measuring the degree of forward displacement of the eye. The device allows measurement of the forward distance of the lateral orbital rim to the front of the cornea. Computed tomography (CT) and magnetic resonance imaging (MRI) may also be used to evaluate the degree of proptosis in a patient.

Orbital imaging can also provide information regarding the amount and distribution of orbital tissue expansion (extraocular muscle thickening, orbital fat volume increases, and lacrimal glands). CT scans allow visualization of enlarged extraocular muscles, orbital fat compartment, and levator enlargement as a source of eyelid retraction as well as the orbital bone architecture. CT scans are often used for assessment of orbital decompression surgery. MRI provides excellent soft tissue resolution allowing better imaging of the optic nerve, orbital fat, and extraocular muscles. MRI can also identify edema within extraocular muscles and surrounding fat tissue on T2-weighted scans. Using T1-weighted MRI scans, the volumes of extraocular muscles (e.g. inferior rectus, superior rectus, medial rectus, and lateral rectus) and intraorbital fat can be assessed using 3-dimensional analysis. In some embodiments, a patient to be treated according to the method of the invention may have increased extraocular muscle volume and/or orbital fat volume prior to administration of the IGF-1R antagonist antibody (i.e. at baseline) as compared to a patient's prior measurements (e.g. prior to the diagnosis of TED) or as compared to volumes in the normal range for the patient's race and gender.

In certain embodiments, patients to be treated according to the methods of the invention have not previously had orbital irradiation or orbital surgeries. For instance, in one embodiment, a patient to be treated according to the methods of the invention has not previously had orbital irradiation. In another embodiment, a patient to be treated according to the methods of the invention has not previously had orbital decompression surgery. In yet another embodiment, a patient to be treated according to the methods of the invention has not previously had strabismus surgery.

In one aspect, the methods of the invention comprise administering to a patient an effective amount of an IGF-1R antagonist antibody. An “effective amount” refers to an amount sufficient to treat, reduce, or ameliorate thyroid eye disease or one or more symptoms of thyroid eye disease, particularly a state or symptoms associated with thyroid eye disease, or otherwise prevent, hinder, retard, or reverse the progression of thyroid eye disease in any way whatsoever. An effective amount can also refer to an amount sufficient to reduce the occurrence or severity of sequelae resulting from thyroid eye disease. For instance, in some embodiments, an effective amount of an IGF-1R antagonist antibody is an amount sufficient to reduce the severity of thyroid eye disease by, for example, reducing proptosis, the severity of diplopia, extraocular muscle volume, orbital fat volume, and/or inflammation of the eye or surrounding tissues.

In certain embodiments of the methods of the invention, an IGF-1R antagonist antibody is administered to a patient at a fixed dose. A “fixed dose” refers to a dose that is administered to all patients regardless of patient-specific factors, such as weight, body size, age, gender, race, ethnicity, and the like. Thus, a fixed dose is not adjusted from patient to patient based on the patient's weight. In some embodiments of the methods of the invention, the IGF-1R antagonist antibody may be administered to a patient at a fixed dose from about 1,500 mg to about 2,000 mg once every two weeks. For instance, the fixed dose of an IGF-1R antagonist antibody can be about 1,500 mg, about 1,525 mg, about 1,550 mg, about 1,575 mg, about 1,600 mg, about 1,625 mg, about 1,650 mg, about 1,675 mg, about 1,700 mg, about 1,725 mg, about 1,750 mg, about 1,775 mg, about 1,800 mg, about 1,825 mg, about 1,850 mg, about 1,875 mg, about 1,900 mg, about 1,925 mg, about 1,950 mg, about 1,975 mg, or about 2,000 mg, wherein the doses are administered once every two weeks. Ranges between any and all of these endpoints are also contemplated, for example, the fixed dose of the IGF-1R antagonist antibody administered to a patient according to the methods of the invention may be from about 1,500 mg to about 1,650 mg, from about 1,675 mg to about 1,850 mg, from about 1,575 mg to about 1,750 mg, from about 1,850 mg to about 1,950 mg, or from about 1,750 mg to about 2,000 mg, wherein the doses are administered once every two weeks. In certain embodiments of the methods of the invention, the IGF-1R antagonist antibody is administered to a patient at a fixed dose from about 1,575 mg to about 1,750 mg once every two weeks. In one preferred embodiment, the IGF-1R antagonist antibody is administered to a patient at a fixed dose of about 1,575 mg once every two weeks. In another preferred embodiment, the IGF-1R antagonist antibody is administered to a patient at a fixed dose of about 1,750 mg once every two weeks.

In other embodiments of the methods of the invention, the fixed dose of an IGF-1R antagonist antibody may be administered on a weekly dosing interval. In such embodiments, the fixed dose of the IGF-1R antagonist antibody may be from about 700 mg to about 1,200 mg, wherein the fixed dose is administered to the patient once per week. A fixed dose of an IGF-1R antagonist antibody suitable for weekly administration can be about 700 mg, about 725 mg, about 750 mg, about 775 mg, about 800 mg, about 825 mg, about 850 mg, about 875 mg, about 900 mg, about 925 mg, about 950 mg, about 975 mg, about 1,000 mg, about 1,025 mg, about 1,050 mg, about 1,075 mg, about 1,100 mg, about 1,125 mg, about 1,150 mg, about 1,175 mg, or about 1,200 mg. Ranges between any and all of these endpoints are also contemplated, such as from about 775 mg to about 900 mg, from about 925 mg to about 1,050 mg, from about 800 mg to about 975 mg, or from about 1,050 mg to about 1,200 mg, wherein the doses are administered once per week. In one embodiment, the IGF-1R antagonist antibody is administered to a patient at a fixed dose of about 800 mg once per week. In another embodiment, the IGF-1R antagonist antibody is administered to a patient at a fixed dose of about 850 mg once per week.

In some embodiments of the methods of the invention, the IGF-1R antagonist antibody is administered to the patient over the course of a set treatment period. A “treatment period” begins upon administration of a first dose of the IGF-1R antagonist antibody and ends upon administration of a final dose of the IGF-1R antagonist antibody. The treatment period may comprise from about 12 weeks to about 48 weeks, such as about 14 weeks, about 16 weeks, about 18 weeks, about 20 weeks, about 22 weeks, about 24 weeks, about 26 weeks, about 28 weeks, about 30 weeks, about 32 weeks, about 34 weeks, about 36 weeks, about 38 weeks, about 40 weeks, about 42 weeks, about 44 weeks, or about 46 weeks. In certain embodiments, the treatment period is 24 weeks—i.e. the IGF-1R antagonist antibody is administered over the course of 24 weeks. In other embodiments, the treatment period is 12 weeks. In yet other embodiments, the treatment period is 36 weeks. In still other embodiments, the treatment period is 48 weeks. In embodiments in which the dosing interval is two weeks, the patient may receive between about 6 injections to about 24 injections depending on the treatment period. In one particular embodiment of the methods of the inventions, the patient receives 12 injections of the IGF-1R antagonist antibody over the course of 24 weeks. In certain embodiments, the IGF-1R antagonist antibody is administered for a treatment period of at least 12 weeks and produces a statistically significant reduction from baseline in proptosis in at least one eye in the patient as compared to patients not receiving the IGF-1R antagonist antibody. In other embodiments, the IGF-1R antagonist antibody is administered for a treatment period of at least 24 weeks and produces a statistically significant reduction from baseline in proptosis in at least one eye in the patient as compared to patients not receiving the IGF-1R antagonist antibody.

The methods of the invention can be used to treat patients for TED who failed to respond to a prior treatment with a therapeutic agent for TED or relapsed after receiving the prior treatment. The prior therapeutic agent may be another IGF-1R inhibitor or an agent with a different mechanism of action. For instance, in one embodiment, the prior therapeutic agent is a glucocorticoid, such as methylprednisolone, prednisone or prednisolone. In another embodiment, the prior therapeutic agent is a CD20 antibody, such as rituximab. In yet another embodiment, the prior therapeutic agent is an interleukin 6 (IL-6) antibody (e.g. siltuximab and olokizumab) or IL-6 receptor antibody (e.g. tocilizumab sarilumab, or satralizumab). In still another embodiment, the prior therapeutic agent is a neonatal Fc receptor (FcRn) inhibitor, such as batoclimab or efgartigimod alfa. In certain embodiments, the prior therapeutic agent is an IGF-1R inhibitor other than the IGF-1R antagonist antibodies described herein, such as linsitinib, lonigutamab, or veligrotug. In certain other embodiments, the prior treatment can be a prior course of the IGR-1R antagonist antibodies described herein. In one such embodiment, the prior treatment is a prior course of teprotumumab.

As used herein, “failure to respond” or “treatment failure” refers to the lack of efficacy of a therapeutic agent in reducing the severity of TED in the patient following a standard therapeutic regimen of the agent. For instance, in one embodiment, a patient who has failed to respond to a prior treatment for TED is a patient who has <2 mm reduction in proptosis following a standard treatment period for the particular therapeutic agent. A patient is considered to have relapsed or experienced a flare when after a period of having responded to a prior treatment, the patient has a recurrence of symptoms of TED. In one embodiment, a patient has relapsed or experienced a flare if the patient has a new onset of diplopia. In another embodiment, a patient has relapsed or experienced a flare if the patient has an increase in proptosis of 2 mm or greater in at least one eye relative to the patient's proptosis measurement at the end of the prior treatment period. In another embodiment, a patient has relapsed or experienced a flare if the patient has a CAS of ≥4 (on either the 7-component or 10-component scale) in at least one eye. In some embodiments, a patient has relapsed or experienced a flare if the patient has a new onset of diplopia and one or both of the following: (i) an increase in proptosis of 2 mm or greater in at least one eye relative to the patient's proptosis measurement at the end of the prior treatment period and (ii) a CAS of ≥4 (on either the 7-component or 10-component scale) in at least one eye with an increase of 2 points or greater in CAS relative to the patient's CAS at the end of the prior treatment period.

In certain embodiments of the methods of the invention, the IGF-1R antagonist antibody is administered at a fixed dose disclosed herein (e.g. about 1,575 mg to about 1,750 mg) once every two weeks over the course of a treatment period to a patient who has failed to respond to a prior treatment with a therapeutic agent for TED. In some such embodiments, the treatment period is 24 weeks (i.e. the patient receives 12 injections of the IGF-1R antagonist antibody). In other such embodiments, the treatment period is 36 weeks (i.e. the patient receives 18 injections of the IGF-1R antagonist antibody). In other embodiments, the treatment period is 48 weeks (i.e. the patient receives 24 injections of the IGF-1R antagonist antibody). In any of the foregoing embodiments, the patient may have failed to respond to a prior treatment with an IGF-1R antagonist antibody described herein (e.g. teprotumumab). In one embodiment, the patient has failed to respond to a prior treatment of teprotumumab given over a treatment period of 24 weeks.

In certain other embodiments of the methods of the invention, the IGF-1R antagonist antibody is administered at a fixed dose disclosed herein (e.g. about 1,575 mg to about 1,750 mg) once every two weeks over the course of a treatment period to a patient who has relapsed or experienced a flare after a prior treatment with a therapeutic agent for TED. In one such embodiment, the treatment period is 12 weeks (i.e. the patient receives 6 injections of the IGF-1R antagonist antibody). In another embodiment, the treatment period is 24 weeks (i.e. the patient receives 12 injections of the IGF-1R antagonist antibody). In yet another embodiment, the treatment period is 36 weeks (i.e. the patient receives 18 injections of the IGF-1R antagonist antibody). In some embodiments, the treatment period is 48 weeks (i.e. the patient receives 24 injections of the IGF-1R antagonist antibody). In any of the foregoing embodiments, the patient may have relapsed or experienced a flare after a prior treatment period with an IGF-1R antagonist antibody described herein (e.g. teprotumumab). In some embodiments, the patient may have relapsed or experienced a flare after a prior treatment period of 24 weeks with teprotumumab.

In some embodiments of the methods of the invention, administration of the IGF-1R antagonist antibody reduces proptosis in the patient as compared to the patient's proptosis measurement prior to administration of the IGF-1R antagonist antibody (i.e. pre-treatment baseline) or as compared to a patient not receiving the IGF-1R antagonist antibody. Thus, the present invention also includes methods of reducing proptosis in a patient with thyroid eye disease comprising administering to the patient a fixed dose regimen of an IGF-1R antagonist antibody, such as any of the fixed dose regimens described herein. Preferably, administration of the IGF-1R antagonist antibody according to the methods of the invention reduces proptosis by at least 2 mm in at least one eye of the patient. In certain embodiments, the reduction in proptosis in the patient may be greater than 2 mm. For example, administration of the IGF-1R antagonist antibody according to the methods of the invention may reduce proptosis in at least one eye of the patient by about 2.5 mm to about 8 mm, about 2.7 mm to about 4.8 mm, about 2.5 mm to about 4 mm, about 2.7 mm to about 6 mm, about 3 mm to about 5 mm, about 4 mm to about 6 mm, or about 4.5 mm to about 7.5 mm. In some embodiments, the reduction in proptosis of the patient may be about 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.2 mm, 5.5 mm, 5.7 mm, 6 mm, 6.2 mm, 6.5 mm, 6.7 mm, 7 mm, 7.2 mm, 7.5 mm, 7.7 mm or 8 mm. In one embodiment, administration of the IGF-1R antagonist antibody according to the methods of the invention reduces proptosis in at least one eye of the patient from about 2.5 mm to about 4 mm. In another embodiment, administration of the IGF-1R antagonist antibody according to the methods of the invention reduces proptosis by at least 3 mm in at least one eye of the patient. In another embodiment, administration of the IGF-1R antagonist antibody according to the methods of the invention reduces proptosis by at least 4 mm in at least one eye of the patient. In one particular embodiment, administration of the IGF-1R antagonist antibody according to the methods of the invention reduces proptosis in at least one eye of the patient to the same degree as orbital decompression surgery (e.g. about 2.7 mm to about 6 mm). Proptosis can be measured clinically with an exophthalmometer (e.g. Hertel exophthalmometer) or radiologically with CT or other imaging techniques as described above.

In other embodiments of the methods of the invention, administration of the IGF-1R antagonist antibody reduces the severity of diplopia in the patient as compared to the patient's degree of diplopia prior to administration of the IGF-1R antagonist antibody (i.e. pre-treatment baseline) or as compared to a patient not receiving the IGF-1R antagonist antibody. Accordingly, the present invention also includes methods of reducing the severity of diplopia in a patient with thyroid eye disease comprising administering to the patient a fixed dose regimen of an IGF-1R antagonist antibody, such as any of the fixed dose regimens described herein. The severity of diplopia can be assessed using the Gorman scale as described above. The patient may have intermittent diplopia (Gorman score 1), inconstant diplopia (Gorman score 2), or constant diplopia (Gorman score 3) at the pre-treatment baseline. In certain embodiments, administration of the IGF-1R antagonist antibody according to the methods of the invention reduces diplopia in the patient by at least one grade on the Gorman scale. In certain other embodiments, administration of the IGF-1R antagonist antibody according to the methods of the invention reduces diplopia in the patient by at least two grades on the Gorman scale. In other embodiments, administration of the IGF-1R antagonist antibody according to the methods of the invention reduces diplopia in the patient by three grades on the Gorman scale. In one particular embodiment, administration of the IGF-1R antagonist antibody according to the methods of the invention eliminates diplopia in the subject—i.e. patient has a Gorman score 0 (complete resolution of diplopia) following administration of the IGF-1R antagonist antibody.

In some embodiments of the methods of the invention, administration of the IGF-1R antagonist antibody reduces the clinical activity score (CAS) in the patient as compared to the patient's CAS prior to administration of the IGF-1R antagonist antibody (i.e. pre-treatment baseline) or as compared to a patient not receiving the IGF-1R antagonist antibody. Thus, the present invention also contemplates methods of reducing the CAS in a patient with thyroid eye disease comprising administering to the patient a fixed dose regimen of an IGF-1R antagonist antibody, such as any of the fixed dose regimens described herein. In preferred embodiments, administration of the IGF-1R antagonist antibody according to the methods of the invention reduces the CAS of the patient by at least two points. In other preferred embodiments, administration of the IGF-1R antagonist antibody according to the methods of the invention reduces the CAS of the patient by at least three points. In certain embodiments of the methods of the invention, the patient has a CAS of 0 or 1 following administration of the IGF-1R antagonist antibody. In related embodiments, administration of the IGF-1R antagonist antibody according to the methods of the invention causes remission of active TED to inactive TED in the patient. CAS can be determined using either the 7-component or 10-component scale described herein. In certain embodiments, the CAS is determined using the 7-component scale.

In other embodiments of the methods of the invention, administration of the IGF-1R antagonist antibody increases motility of at least one eye of the patient as compared to the motility of the eye prior to administration of the IGF-1R antagonist antibody (i.e. pre-treatment baseline) or as compared to a patient not receiving the IGF-1R antagonist antibody. Accordingly, the present invention also encompasses methods of increasing motility of at least one eye of a patient with thyroid eye disease comprising administering to the patient a fixed dose regimen of an IGF-1R antagonist antibody, such as any of the fixed dose regimens described herein. Eye motility in the patient can be assessed using the light reflex test described in Dolman et al., Ophthalmology, Vol. 119 (2): 382-389, 2012. The light reflex test entails a clinician shining a pen light in line with the eye being examined in ambient room light and observes the participant's eye along the light's axis. The participant is asked to look in the 4 cardinal directions and the position of the light reflex is viewed on the surface of the cornea. If the light touches the limbus, the eye is assessed as being turned 45 degrees; if halfway between the limbus and pupil edge, the eye is assessed as being at 30 degrees; and if it is at the pupil edge, it is assessed as being at 15 degrees. Intermediate ductions are judged by estimating the light position between these points to the nearest 5 degrees. The monocular ductions of each eye (degrees) are recorded for adduction, abduction, supraduction and infraduction. In particular embodiments, administration of the IGF-1R antagonist antibody according to the methods of the invention increases eye motility by ≥8° duction in at least one direction of gaze.

In certain embodiments of the methods of the invention, administration of the IGF-1R antagonist antibody reduces extraocular muscle volume and/or orbital fat volume in at least one eye of the patient as compared to the patient's extraocular muscle and orbital fat volumes prior to administration of the IGF-1R antagonist antibody (i.e. pre-treatment baseline) or as compared to a patient not receiving the IGF-1R antagonist antibody. Therefore, the present invention also includes methods of reducing extraocular muscle volume and/or orbital fat volume in at least one eye of a patient with thyroid eye disease comprising administering to the patient a fixed dose regimen of an IGF-1R antagonist antibody, such as any of the fixed dose regimens described herein. As described above, extraocular muscles and orbital fat can be visualized using various imaging techniques, such as CT and MRI, and volumes of the extraocular muscle and orbital fat can be calculated from three-dimensional analysis of the images (see, e.g., Jain et al., Br. J. Ophthalmol., Vol. 106(2):165-171, 2022). In some embodiments, the volume of the inferior rectus, superior rectus, medial rectus, and lateral rectus muscles are measured. In such embodiments, administration of the IGF-1R antagonist antibody according to the methods of the invention reduces the total extraocular muscle volume (i.e. total volume of all four muscles measured) in at least one eye of the patient by at least 10%, for example, by about 15%, about 20%, about 25%, about 30%, about 35%, about 40%, about 45%, about 50%, about 55%, or about 60% or more. In one embodiment, administration of the IGF-1R antagonist antibody according to the methods of the invention reduces the total extraocular muscle volume in at least one eye by at least 25%. In another embodiment, administration of the IGF-1R antagonist antibody according to the methods of the invention reduces the total extraocular muscle volume in at least one eye by at least 30%. In yet another embodiment, administration of the IGF-1R antagonist antibody according to the methods of the invention reduces the total extraocular muscle volume in at least one eye by at least 35%. In some embodiments, administration of the IGF-1R antagonist antibody according to the methods of the invention reduces the total extraocular muscle volume in at least one eye from about 15% to about 65%, from about 20% to about 40%, from about 25% to about 45%, or from about 30% to about 60%.

In certain other embodiments, administration of the IGF-1R antagonist antibody according to the methods of the invention reduces the orbital fat volume in at least one eye of the patient by at least 5%, for example, by about 7%, about 10%, about 15%, about 20%, about 25%, about 30%, about 35%, about 40%, about 45%, about 50%, or about 55% or more. In one embodiment, administration of the IGF-1R antagonist antibody according to the methods of the invention reduces the orbital fat volume in at least one eye by at least 30%. In another embodiment, administration of the IGF-1R antagonist antibody according to the methods of the invention reduces the orbital fat volume in at least one eye by at least 35%. In yet another embodiment, administration of the IGF-1R antagonist antibody according to the methods of the invention reduces the orbital fat volume in at least one eye by at least 40%. In some embodiments, administration of the IGF-1R antagonist antibody according to the methods of the invention reduces the orbital fat volume in at least one eye from about 5% to about 55%, from about 15% to about 50%, from about 30% to about 45%, or from about 25% to about 40%.

In some embodiments of the methods of the invention, administration of the IGF-1R antagonist antibody improves one or more aspects of the quality of life of the patient as compared to such aspects of the patient's quality of life prior to administration of the IGF-1R antagonist antibody (i.e. pre-treatment baseline) or as compared to a patient not receiving the IGF-1R antagonist antibody. Thus, the present invention provides methods of improving the quality of life of a patient with thyroid eye disease comprising administering to the patient a fixed dose regimen of an IGF-1R antagonist antibody, such as any of the fixed dose regimens described herein. A patient's quality of life can be measured by a number of patient reported outcome tools, such as EuroQol 5-Dimension 5-level (EQ-5D-5L) questionnaire, Hospital Anxiety and Depression Scale (HADS), the Work Productivity and Activity Impairment (WPAI) questionnaire, visual analog scale (VAS) to assess pain intensity, and the Graves' ophthalmopathy quality of life (GO-QoL) questionnaire described herein. In certain embodiments, administration of the IGF-1R antagonist antibody according to the methods of the invention improves visual function in the patient, for example as measured by the visual function subscale of the GO-QoL questionnaire. In such embodiments, administration of the IGF-1R antagonist antibody results in a change (increase indicates improvement) of at least 8 points on the visual function subscale of the GO-QoL questionnaire, such as an increase of about 9 points, about 10 points, about 11 points, about 12 points, about 13 points, about 14 points, or about 15 points or more. In one embodiment, administration of the IGF-1R antagonist antibody results in an increase of at least 10 points on the visual function subscale of the GO-QoL questionnaire. In another embodiment, administration of the IGF-1R antagonist antibody results in an increase of at least 12 points on the visual function subscale of the GO-QoL questionnaire. In yet another embodiment, administration of the IGF-1R antagonist antibody results in an increase of at least 15 points on the visual function subscale of the GO-QoL questionnaire.

In certain other embodiments, administration of the IGF-1R antagonist antibody according to the methods of the invention improves visual appearance in the patient, for example as measured by the visual appearance subscale of the GO-QoL questionnaire. In such embodiments, administration of the IGF-1R antagonist antibody results in a change (increase indicates improvement) of at least 8 points on the visual appearance subscale of the GO-QoL questionnaire, such as an increase of about 9 points, about 10 points, about 11 points, about 12 points, about 13 points, about 14 points, about 15 points, about 16 points, about 17 points, about 18 points, about 19 points, about 20 points, about 21 points, about 22 points, about 23 points, about 24 points, or about 25 points or more. In one embodiment, administration of the IGF-1R antagonist antibody results in an increase of at least 10 points on the visual appearance subscale of the GO-QoL questionnaire. In another embodiment, administration of the IGF-1R antagonist antibody results in an increase of at least 14 points on the visual appearance subscale of the GO-QoL questionnaire. In yet another embodiment, administration of the IGF-1R antagonist antibody results in an increase of at least 20 points on the visual appearance subscale of the GO-QoL questionnaire. In still another embodiment, administration of the IGF-1R antagonist antibody results in an increase of at least 25 points on the visual appearance subscale of the GO-QoL questionnaire.

Administration of the IGF-1R antagonist antibody according to the methods of the invention to a patient with TED can delay or eliminate the need for a more invasive treatment, such as orbital irradiation, orbital decompression surgery, or strabismus surgery. In some embodiments, treatment of a TED patient according to the methods of the invention may improve the outcomes of the patient following a surgical procedure, such as orbital decompression surgery or strabismus surgery.

The methods described herein comprise administering to a patient an IGF-1R antagonist antibody. As used herein, the term “IGF-1R antagonist antibody” refers to an antibody that specifically binds to human IGF-1R and inhibits its activation and downstream signaling by any of its ligands (e.g. IGF-1 and IGF-2). An “antibody” generally refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). The antibody chains are linked together via inter-polypeptide disulfide bonds between the CL domain and the CHi domain (i.e. between the light and heavy chain) and between the hinge regions of the two antibody heavy chains. Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The antibodies suitable for use in the methods of the invention can be any of these immunoglobulin isotypes, including subtypes. In certain embodiments, the antibody used in the methods of the invention is an IgG antibody, including IgG1, IgG2, IgG3, and IgG4 antibodies. In one particular embodiment, the antibody used in the methods of the invention is an IgG1 antibody. In another particular embodiment, the antibody used in the methods of the invention is an IgG2 antibody.

An antibody “specifically binds” to a target antigen when it has a significantly higher binding affinity for, and consequently is capable of distinguishing, that antigen compared to its affinity for other unrelated proteins, under similar binding assay conditions. Antibodies that specifically bind an antigen may have an equilibrium dissociation constant (K_{D})≤1×10⁻⁶ M. The antibody specifically binds antigen with “high affinity” when the K_{D} is ≤1×10⁻⁸ M. In one embodiment, the antibodies suitable for use in the methods of the invention bind to human IGF-1R with a K_{D} of 5×10⁻⁹ M. In another embodiment, the antibodies suitable for use in the methods of the invention bind to human IGF-1R with a K_{D} of ≤1×10⁻⁹ M. In yet another embodiment, the antibodies suitable for use in the methods of the invention bind to human IGF-1R with a K_{D} of ≤5×10⁻¹⁰ M.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/k^{a}). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (k_{d} in s⁻¹) can be calculated from these values (K_{D}×kₐ). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (kₐ and k_{d}) and affinity (K_{D}) constants can be calculated in real-time using the bio-layer interferometry method. In some embodiments, the antibodies suitable for use in the methods of the invention exhibit desirable characteristics such as binding avidity as measured by k_{d} (dissociation rate constant) for human IGF-1R of about 10⁻², 10⁻³, 10⁻⁴, 10⁻⁵, 10⁻⁶, 10⁻⁷, 10⁻⁸, 10⁻⁹, 10⁻¹⁰ s⁻¹ or lower (lower values indicating higher binding avidity), and/or binding affinity as measured by K_{D} (equilibrium dissociation constant) for human IGF-1R of about 10⁻⁸, 10⁻⁹, 10⁻¹⁰, 10⁻¹¹, 10⁻¹² M or lower (lower values indicating higher binding affinity).

The inhibitory activity of an IGF-1R antagonist antibody can be assessed by assays measuring the inhibition of binding of IGF-1 and IGF-2 to the IGF-1R by the antibody. Preferably, the IGF-1R antagonist antibody inhibits the binding of IGF-1 and/or IGF-2 to the IGF-1R with an IC50 value less than 2 nM and more preferably with an IC50 value less than 1 nM. Inhibitory activity of an IGF-1R antagonist antibody can also be assessed by in vitro assays measuring the ligand-induced phosphorylation of the IGF-1R or phosphorylation of components of its downstream signaling cascade (e.g. phosphorylation of AKT/PKB) or by assays measuring the downregulation of cell surface expressed IGF-1R induced by the antibody. The IC50 or EC50 values for IGF-1R antagonist antibodies suitable for use in the methods of the invention in any of these assays are preferably less than 1 nM with max activity greater than 75%. All of the foregoing assays for assessing IGF-1R inhibition by various molecules as well as other assays are known to those of skill in the art, such as those described in U.S. Pat. No. 7,579,157 and WO 2023/237928.

IGF-1R antagonist antibodies for use in the methods of the invention comprise a heavy chain variable region comprising a CDRH1, CDRH2, and CDRH3, and a light chain variable region comprising a CDRL1, CDRL2, and CDRL3. The term “CDR” refers to the complementarity determining region (also termed “minimal recognition units” or “hypervariable region”) within antibody variable sequences. There are three heavy chain variable region CDRs (CDRH1, CDRH2 and CDRH3) and three light chain variable region CDRs (CDRL1, CDRL2 and CDRL3). The term “CDR region” as used herein refers to a group of three CDRs that occur in a single variable region (i.e. the three light chain CDRs or the three heavy chain CDRs). The CDRs in each of the two chains typically are aligned by the framework regions (FRs) to form a structure that binds specifically with a specific epitope or domain on the target protein (e.g., human IGF-1R). From N-terminus to C-terminus, naturally-occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309(3):657-670; 2001).

The “variable region,” used interchangeably herein with “variable domain” (variable region of a light chain (VL), variable region of a heavy chain (VH)), refers to the region in each of the light and heavy immunoglobulin chains which is involved directly in binding the antibody to the antigen. As discussed above, the regions of variable light and heavy chains have the same general structure and each region comprises four framework (FR) regions, the sequences of which are widely conserved, connected by three CDRs. The framework regions adopt a beta-sheet conformation and the CDRs may form loops connecting the beta-sheet structure. The CDRs in each chain are held in their three-dimensional structure by the framework regions and form, together with the CDRs from the other chain, the antigen binding site.

In certain embodiments, the IGF-1R antagonist antibody administered to a patient according to the methods of the invention comprises (i) a heavy chain variable region comprising a CDRH1 having the sequence of SEQ ID NO: 1, a CDRH2 having the sequence of SEQ ID NO: 2, and a CDRH3 having the sequence of SEQ ID NO: 3, and (ii) a light chain variable region comprising a CDRL1 having the sequence of SEQ ID NO: 4, a CDRL2 having the sequence of SEQ ID NO: 5, and a CDRL3 having the sequence of SEQ ID NO: 6. In some such embodiments, the IGF-1R antagonist antibody administered to a patient according to the methods of the invention comprises a heavy chain variable region comprising: (i) a sequence that is at least 90% identical to the sequence of SEQ ID NO: 7, (ii) a sequence that is at least 95% identical to the sequence of SEQ ID NO: 7, or (iii) the sequence of SEQ ID NO: 7. In related embodiments, the IGF-1R antagonist antibody administered to a patient according to the methods of the invention comprises a light chain variable region comprising: (i) a sequence that is at least 90% identical to the sequence of SEQ ID NO: 8, (ii) a sequence that is at least 95% identical to the sequence of SEQ ID NO: 8, or (iii) the sequence of SEQ ID NO: 8. In certain embodiments, the IGF-1R antagonist antibody administered to a patient according to the methods of the invention comprises a heavy chain variable region comprising the sequence of SEQ ID NO: 7, and a light chain variable region comprising the sequence of SEQ ID NO: 8.

The term “identity,” as used herein, refers to a relationship between the sequences of two or more polypeptide molecules. “Percent identity,” as used herein, means the percent of identical residues between the amino acids in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (Dayhoff et al., in Atlas of Protein Sequence and Structure, vol. 5, supp. 3, 1978) or BLOSUM62 (Henikoff et al., 1992, Proc. Natl. Acad. Sci. U.S.A. 89:10915-10919) can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, WI). The computer algorithm GAP is used to align the two polypeptides for which the percent sequence identity is to be determined. The sequences are aligned for optimal matching of their respective amino acid sequences (the “matched span,” as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides sequences using the GAP program include the following:
Algorithm: Needleman et al. 1970, J. Mol. Biol. 48:443-453;
Comparison matrix: BLOSUM 62 from Henikoff et al., 1992, supra;
Gap Penalty: 12 (but with no penalty for end gaps)
Gap Length Penalty: 4
Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

In some embodiments, the IGF-1R antagonist antibodies for use in the methods of the invention are monoclonal antibodies. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against an individual antigenic site or epitope, in contrast to polyclonal antibody preparations that typically include different antibodies directed against different epitopes. Monoclonal antibodies may be produced using any technique known in the art, e.g., by immortalizing spleen cells harvested from an animal after completion of the immunization schedule. Another useful method for producing monoclonal antibodies is the SLAM method described in Babcook et al., Proc. Natl. Acad. Sci. USA, Vol. 93: 7843-7848, 1996. Preferably, the monoclonal IGF-1R antagonist antibodies are human monoclonal antibodies or humanized monoclonal antibodies.

In certain embodiments, the IGF-1R antagonist antibodies for use in the methods of the invention are human antibodies, particularly, human monoclonal antibodies. A “human antibody,” or “fully human antibody” is an antibody that comprises variable and constant regions derived from or indicative of human germ line immunoglobulin sequences. One specific means provided for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (mAbs) in mouse, an animal that can be immunized with any desirable antigen. One particular transgenic mouse line suitable for generation of fully human antibodies is the XenoMouse® transgenic mouse line described in U.S. Pat. Nos. 6,114,598; 6,162,963; 6,833,268; 7,049,426; 7,064,244; Green et al., 1994, Nature Genetics 7:13-21; Mendez et al., 1997, Nature Genetics 15:146-156; Green and Jakobovitis, 1998, J. Ex. Med, 188:483-495; Green, 1999, Journal of Immunological Methods 231:11-23; Kellerman and Green, 2002, Current Opinion in Biotechnology 13, 593-597. Using fully human antibodies can minimize the immunogenic and allergic responses that can sometimes be caused by administering mouse or mouse-derived antibodies to humans as therapeutic agents. Human monoclonal antibodies can be produced by generating hybridomas from transgenic animals (usually mice) that are capable of producing a repertoire of human antibodies that have been immunized with a particular antigen. Human-derived antibodies can also be generated using phage display techniques. Phage display is described in e.g., Dower et al., WO 91/17271, McCafferty et al., WO 92/01047, and Caton and Koprowski, 1990, Proc. Natl. Acad. Sci. USA, 87:6450-6454.

In some embodiments, the IGF-1R antagonist antibodies for use in the methods of the invention are humanized antibodies, particularly, humanized monoclonal antibodies. The term “humanized antibody” as used herein refers to antibodies in which one or more CDRs from a particular species or belonging to a particular antibody class or subclass are grafted into a human antibody, replacing the naturally-occurring CDRs of the human antibody. Generally, a humanized antibody is produced from a monoclonal antibody raised initially in a non-human animal, such as a rodent or rabbit. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent or rabbit variable region for the corresponding regions of a human antibody (see, e.g., U.S. Pat. Nos. 5,585,089, and 5,693,762; Jones et al., 1986, Nature 321:522-525; Riechmann et al., 1988, Nature 332:323-27; and Verhoeyen et al., 1988, Science 239:1534-1536). In some embodiments, the variable region or selected CDRs from a human antibody may be grafted into another human antibody from a different antibody class or subclass.

In certain embodiments, the IGF-1R antagonist antibody administered to a patient according to the methods of the invention is a human monoclonal antibody. In such embodiments, the IGF-1R antagonist antibody administered to a patient according to the methods of the invention comprises a heavy chain comprising: (i) a sequence that is at least 90% identical to the sequence of SEQ ID NO: 9, (ii) a sequence that is at least 95% identical to the sequence of SEQ ID NO: 9, or (iii) the sequence of SEQ ID NO: 9. In related embodiments, the IGF-1R antagonist antibody administered to a patient according to the methods of the invention comprises a light chain comprising: (i) a sequence that is at least 90% identical to the sequence of SEQ ID NO: 10, (ii) a sequence that is at least 95% identical to the sequence of SEQ ID NO: 10, or (iii) the sequence of SEQ ID NO: 10. In some embodiments, the IGF-1R antagonist antibody administered to a patient according to the methods of the invention comprises a heavy chain comprising the sequence of SEQ ID NO: 9, and a light chain comprising the sequence of SEQ ID NO: 10. In other embodiments, the IGF-1R antagonist antibody administered to a patient according to the methods of the invention comprises a heavy chain comprising the sequence of SEQ ID NO: 11, and a light chain comprising the sequence of SEQ ID NO: 10.

In certain other embodiments, the IGF-1R antagonist antibody administered to a patient according to the methods of the invention is teprotumumab. The term “teprotumumab” refers a human monoclonal IgG1 antibody comprising the heavy chain variable region sequence of SEQ ID NO: 7 and the light chain variable region sequence of SEQ ID NO: 8. The heavy chain variable region sequence of SEQ ID NO: 7 comprises CDRH1, CDRH2, and CDRH3 sequences of SEQ ID NOs: 1-3, respectively. The light chain variable region sequence of SEQ ID NO: 8 comprises CDRL1, CDRL2, and CDRL3 sequences of SEQ ID NOs: 4-6, respectively. In one embodiment, teprotumumab comprises a heavy chain comprising the sequence of SEQ ID NO: 9 and a light chain comprising the sequence of SEQ ID NO: 10. In such embodiments, teprotumumab is an antibody comprising two heavy chains and two light chains, wherein each of the heavy chains comprises the sequence of SEQ ID NO: 9 and each of the light chains comprises the sequence of SEQ ID NO: 10. When produced recombinantly, teprotumumab can undergo common post-translational modifications at the termini of the heavy chains, such as removal of the C-terminal lysine residue at position 448 from the heavy chain and cyclization of the N-terminal glutamine residue in the heavy chain to pyroglutamate. Thus, the term teprotumumab can also refer to an IgG1 antibody that lacks the C-terminal lysine residue in one or both of the heavy chains and/or comprises a pyroglutamate residue as the N-terminal residue in place of the glutamine residue in one or both of the heavy chains. For instance, in some embodiments, teprotumumab is an antibody comprising two heavy chains and two light chains, wherein each of the heavy chains comprises the sequence of SEQ ID NO: 11 and each of the light chains comprises the sequence of SEQ ID NO: 10. In other embodiments, teprotumumab is an antibody comprising two heavy chains and two light chains, wherein each of the heavy chains comprises the sequence of SEQ ID NO: 9 or SEQ ID NO: 11 and each of the light chains comprises the sequence of SEQ ID NO: 10.

Other IGF-1R antagonist antibodies that could be used in the methods of the invention include, but are not limited to, lonigutamab, veligrotug, ganitumab, figitumumab, cixutumumab, dalotuzumab, robatumumab, and antibodies described in WO2005/061541, WO2006/069202, WO2015/162292, WO2022/081799, WO2022/081804, WO2023/133485, WO2023/133486, and WO2023/122714.

The IGF-1R antagonist antibodies for use in the methods of the invention may be prepared by any of a number of conventional techniques. For example, the IGF-1R antagonist antibodies may be produced by recombinant expression systems using one or more expression vectors comprising polynucleotides encoding the heavy chain and light chain of the antibodies. The polynucleotide encoding the heavy chain and the polynucleotide encoding the light chain of the IGF-1R antagonist antibody can be inserted into a single expression vector or they can be inserted into separate expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Vectors may also include one or more selectable marker genes to facilitate selection of host cells into which the vectors have been introduced.

After the expression vector has been constructed and the one or more nucleic acid molecules encoding the heavy and light chain components of an IGF-1R antagonist antibody has been inserted into the proper site(s) of the vector or vectors, the completed vector(s) may be inserted into a suitable host cell for amplification and/or polypeptide expression. The transformation of an expression vector for the IGF-1R antagonist antibody into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989).

A host cell, when cultured under appropriate conditions, synthesizes the IGF-1R antagonist antibody, which can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. lichenformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antibodies can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antibodies from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, CHO-DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, and a number of other cell lines. CHO cells are preferred host cells in some embodiments for expressing the IGF-1R antagonist antibody.

Host cells are transformed or transfected with the above-described expression vectors for production of the IGF-1R antagonist antibody and are cultured in conventional nutrient media modified as appropriate for inducing promoters, selecting transformants, or amplifying the genes encoding the desired sequences. The host cells used to produce the IGF-1R antagonist antibody may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, any of the media described in Ham et al., Meth. Enz. 58: 44, 1979; Barnes et al., Anal. Biochem. 102: 255, 1980; U.S. Pat. Nos. 4,767,704; 4,657,866; 4,927,762; 4,560,655; or 5,122,469; WO 90/03430; or WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinary skilled artisan.

Upon culturing the host cells, the antibody can be produced intracellularly, in the periplasmic space, or directly secreted into the medium. If the antibody is produced intracellularly, as a first step, the host cells are lysed (e.g., by mechanical shear, osmotic shock, or enzymatic methods) and the particulate debris (e.g., host cells and lysed fragments), is removed, for example, by centrifugation, microfiltration, or ultrafiltration. If the antibody is secreted into the culture medium, the antibody can be separated from host cells through centrifugation or microfiltration, and optionally, subsequently concentrated through ultrafiltration. The IGF-1R antagonist antibody can be further purified or partially purified using, for example, one or more chromatography steps, such as affinity chromatography (e.g. protein A or protein G affinity chromatography), cation exchange chromatography, anion exchange chromatography, hydroxyapatite chromatography, hydrophobic interaction chromatography, or mixed mode chromatography.

The IGF-1R antagonist antibody is generally administered to the patient in a pharmaceutical composition, which can include one or more pharmaceutically acceptable excipients. “Pharmaceutically-acceptable” refers to molecules, compounds, and compositions that are non-toxic to human recipients at the dosages and concentrations employed and/or do not produce allergic or adverse reactions when administered to humans. In certain embodiments, the pharmaceutical formulation may contain materials for modifying, maintaining or preserving, for example, the pH, osmolarity, viscosity, clarity, color, isotonicity, odor, sterility, stability, rate of dissolution or release, adsorption or penetration of the pharmaceutical composition. In such embodiments, suitable materials include, but are not limited to, amino acids (such as glycine, glutamine, asparagine, methionine, histidine, arginine or lysine); antimicrobials; antioxidants (such as ascorbic acid, sodium sulfite or sodium hydrogen-sulfite); buffers (such as acetate, bicarbonate, Tris-HCl, amino acid buffers, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); surfactants or wetting agents (such as pluronics, PEG, sorbitan esters, poloxamers, polysorbates such as polysorbate 20, polysorbate 80, triton, tromethamine, lecithin, cholesterol, tyloxapal); stability enhancing agents (such as trehalose, sucrose or sorbitol); tonicity enhancing agents (such as alkali metal halides, preferably sodium or potassium chloride, mannitol, sorbitol); diluents; excipients and/or pharmaceutical adjuvants. Methods and suitable materials for formulating molecules for therapeutic use are known in the pharmaceutical arts, and are described, for example, in REMINGTON'S PHARMACEUTICAL SCIENCES, 18th Edition, (A. R. Genrmo, ed.), 1990, Mack Publishing Company.

In certain embodiments, the pharmaceutical compositions for use in the methods of the invention comprise the IGF-1R antagonist antibody in concentrations of at least about 100 mg/mL, such as about 125 mg/mL, about 150 mg/mL, about 175 mg/mL, or about 200 mg/mL. In some embodiments, the pharmaceutical composition comprises the IGF-1R antagonist antibody at a concentration of about 150 mg/mL to about 200 mg/mL. In one particular embodiment, the pharmaceutical composition comprises the IGF-1R antagonist antibody at a concentration of about 150 mg/mL. In another particular embodiment, the pharmaceutical composition comprises the IGF-1R antagonist antibody at a concentration of about 200 mg/mL.

In some embodiments, the pharmaceutical compositions for use in the methods of the invention comprise an IGF-1R antagonist antibody described herein, a buffer that maintains the pH of the solution within a range of about 5.0 to about 6.0, a stabilizer, and a surfactant. Suitable buffers include, but are not limited to, glutamate, acetate, Tris, citrate, histidine, succinate, and phosphate buffers. In certain embodiments, the pharmaceutical formulation comprises a histidine buffer, particularly a histidine/histidine hydrocholoride buffer. The histidine buffer can be made by mixing L-histidine and L-histidine hydrocholoride in a specific ratio and amount. Alternatively, the histidine buffer can be prepared by dissolving a specific amount of L-histidine hydrochloride or L-histidine in water and adjusting pH with sodium hydroxide or hydrochloric acid. Pharmaceutical compositions comprising a histidine buffer typically have a pH of about 5.0 to about 7.5 or a pH of about 5.0 to about 6.0, including a pH of about 5.0, about 5.1, about 5.2, about 5.3, about 5.4, about 5.5, about 5.6, about 5.7, about 5.8, about 5.9, and about 6.0.

A stabilizer refers to an excipient that stabilizes the native conformation of the antibody and/or prevents or reduces the physical or chemical degradation of the antibody. Suitable stabilizers include, but are not limited to, polyols (e.g. sorbitol, glycerol, mannitol, xylitol, maltitol, lactitol, erythritol and threitol), sugars (e.g., fructose, glucose, glyceraldehyde, lactose, arabinose, mannose, xylose, ribose, rhamnose, galactose maltose, sucrose, trehalose, sorbose, sucralose, melezitose and raffinose), and amino acids (e.g., glycine, methionine, proline, lysine, arginine, histidine, or glutamic acid). In some embodiments, the pharmaceutical compositions for use in the methods of the invention comprise a sugar as a stabilizer. In these and other embodiments, the sugar is trehalose. In other embodiments, the sugar is sorbitol. In some embodiments, the pharmaceutical compositions for use in the methods of the invention comprise one or more amino acids as a stabilizer. In one such embodiment, the amino acid is arginine. In another embodiment, the amino acid is methionine. In yet another embodiment, the pharmaceutical compositions comprise both arginine and methionine as stabilizers. In certain embodiments, the pharmaceutical compositions for use in the methods of the invention may comprise more than one stabilizer. For instance, in some embodiments, the pharmaceutical compositions may comprise a sugar and an amino acid as stabilizers. In one embodiment, the pharmaceutical compositions comprise trehalose and arginine. In another embodiment, the pharmaceutical compositions comprise trehalose and methionine. In still another embodiment, the pharmaceutical compositions comprise trehalose, arginine, and methionine.

In certain embodiments, the pharmaceutical compositions for use in the methods of the invention comprise a surfactant. A surfactant is a substance that functions to reduce the surface tension of a liquid in which it is dissolved. Surfactants can be included in pharmaceutical compositions for a variety of purposes including, for example, to prevent or control aggregation, particle formation and/or surface adsorption in liquid formulations or to prevent or control these phenomena during the lyophilization and/or reconstitution process in lyophilized formulations. Surfactants include, for example, amphipathic organic compounds that exhibit partial solubility in both organic solvents and aqueous solutions. General characteristics of surfactants include their ability to reduce the surface tension of water, reduce the interfacial tension between oil and water and also form micelles. Surfactants that may be incorporated into the pharmaceutical compositions for use in the methods of the invention include both non-ionic and ionic surfactants. Suitable non-ionic surfactants include, but are not limited to, alkyl poly (ethylene oxide), alkyl polyglucosides, such as octyl glucoside and decyl maltoside, fatty alcohols, such as cetyl alcohol and oleyl alcohol, cocamide MEA, cocamide DEA, and cocamide TEA. Specific examples of non-ionic surfactants include the polysorbates including, for example, polysorbate 20, polysorbate 28, polysorbate 40, polysorbate 60, polysorbate 65, polysorbate 80, polysorbate 81, polysorbate 85 and the like; the poloxamers including, for example, poloxamer 188, also known as poloxalkol or poly(ethylene oxide)-poly(propylene oxide), poloxamer 407 or polyethylene-polypropylene glycol and the like, and polyethylene glycol (PEG). Suitable ionic surfactants include, for example, anionic, cationic and zwitterionic surfactants. Anionic surfactants include, but are not limited to, sulfonate-based or carboxylate-based surfactants such as soaps, fatty acid salts, sodium dodecyl sulfate (SDS), ammonium lauryl sulfate and other alkyl sulfate salts. Cationic surfactants include, but are not limited to, quaternary ammonium-based surfactants such as cetyl trimethylammonium bromide (CTAB), other alkyltrimethylammonium salts, cetyl pyridinium chloride, polyethoxylated tallow amine (POEA) and benzalkonium chloride. Zwitterionic or amphoteric surfactants include, for example, dodecyl betaine, dodecyl dimethylamine oxide, cocamidopropyl betaine and coco ampho glycinate. In certain embodiments, the pharmaceutical compositions for use in the methods of the invention comprise a non-ionic surfactant. In one embodiment, the non-ionic surfactant is polysorbate 20. In another embodiment, the non-ionic surfactant is polysorbate 80. In a preferred embodiment, the non-ionic surfactant is poloxamer 188.

In certain embodiments, the pharmaceutical compositions for use in the methods of the invention comprise any one of the IGF-1R antagonist antibodies described herein (e.g. about 150 mg/mL to about 200 mg/mL of an IGF-1R antagonist antibody), about 15 mM to about 25 mM histidine (e.g. histidine/histidine hydrochloride), about 200 mM to about 275 mM trehalose, and about 0.005% (w/v) to about 0.05% (w/v) polysorbate 20 or polysorbate 80. The pH of these formulations is in the range of about 5.0 to about 6.0 (e.g., pH of about 5.0, about 5.1, about 5.2, about 5.3, about 5.4, about 5.5, about 5.6, about 5.7, about 5.8, about 5.9, or about 6.0). In one particular embodiment, the pharmaceutical composition comprises 150 mg/mL of an IGF-1R antagonist antibody described herein, about 20 mM histidine (e.g. histidine/histidine hydrochloride), about 250 mM trehalose, and about 0.01% (w/v) polysorbate 20, wherein the pharmaceutical composition has a pH of about 5.5±0.5. In another embodiment, the pharmaceutical composition comprises 150 mg/mL of an IGF-1R antagonist antibody described herein, about 20 mM histidine (e.g. histidine/histidine hydrochloride), about 250 mM trehalose, and about 0.01% (w/v) polysorbate 80, wherein the pharmaceutical composition has a pH of about 5.5±0.5. In some embodiments, the pharmaceutical compositions may further comprise another stabilizer, such as methionine or arginine. In one such embodiment, the pharmaceutical composition comprises 150 mg/mL of an IGF-1R antagonist antibody described herein, about 20 mM histidine (e.g. histidine/histidine hydrochloride), about 210 mM trehalose, about 40 mM methionine, and about 0.03% (w/v) polysorbate 20, wherein the pharmaceutical composition has a pH of about 5.5±0.5.

In certain other embodiments, the pharmaceutical compositions for use in the methods of the invention comprise any one of the IGF-1R antagonist antibodies described herein (e.g. about 150 mg/mL to about 200 mg/mL of an IGF-1R antagonist antibody), about 15 mM to about 25 mM histidine (e.g. histidine/histidine hydrochloride), about 200 mM to about 275 mM trehalose, about 30 mM to about 50 mM methionine, and about 0.05% (w/v) to about 0.35% (w/v) poloxamer 188. The pH of these formulations is in the range of about 5.0 to about 6.0 (e.g., pH of about 5.0, about 5.1, about 5.2, about 5.3, about 5.4, about 5.5, about 5.6, about 5.7, about 5.8, about 5.9, or about 6.0). In one particular embodiment, the pharmaceutical composition comprises 150 mg/mL of an IGF-1R antagonist antibody described herein, about 20 mM histidine (e.g. histidine/histidine hydrochloride), about 210 mM trehalose, about 40 mM methionine, and about 0.2% (w/v) poloxamer 188, wherein the pharmaceutical composition has a pH of about 5.5±0.5.

Any of the IGF-1R antagonist antibodies described herein, can be incorporated into any of the pharmaceutical compositions described above and administered to a patient according to the methods described herein. In a preferred embodiment, the IGF-1R antagonist antibody is teprotumumab.

The pharmaceutical compositions are preferably suitable for parenteral injection (e.g. subcutaneous injection). Illustrative pharmaceutical forms suitable for parenteral injection include sterile aqueous solutions or dispersions and sterile powders for the extemporaneous preparation of sterile injectable solutions or dispersions. Preferably, the pharmaceutical composition is sterile and is sufficiently fluid to allow for delivery via a syringe or other injection device (i.e., the composition is not excessively viscous so as to prevent passage through a syringe or other injection device). Sterilization can be accomplished by filtration through sterile filtration membranes. When the composition is lyophilized, sterilization using this filtration method may be conducted either prior to or following lyophilization and reconstitution. Pharmaceutical compositions for parenteral administration can be stored in lyophilized form or in a solution. Parenteral compositions can be placed into a container having a sterile access port, for example, an intravenous solution bag or vial having a stopper pierceable by a hypodermic injection needle. Parenteral compositions can also be stored in syringes, syringe pumps, microinfusion pumps, on-body injection devices, autoinjector devices, or pen injection devices or cartridges adapted for use with such injection devices.

In certain embodiments of the methods of the invention, the IGF-1R antagonist antibodies or pharmaceutical compositions comprising the IGF-1R antagonist antibodies are preferably administered to patients parenterally. Parenteral administration includes intraperitoneal, intramuscular, intravenous, intraarterial, intradermal, subcutaneous, intracerebral, intracerebroventricular, and intrathecal administration. In particular embodiments, the IGF-1R antagonist antibodies or pharmaceutical compositions comprising the IGF-1R antagonist antibodies are administered to patients subcutaneously, for example, by subcutaneous injection. The injections may be delivered to the patients using one or more of the devices (e.g. devices pre-filled with the pharmaceutical compositions) described below.

The fixed doses of the IGF-1R antagonist antibody can be administered at each dosing interval as a single bolus administration (e.g. in a single subcutaneous injection) or as two or more consecutive bolus administrations (e.g. two or more subcutaneous injections). In some embodiments, the entire amount of the fixed dose of the IGF-1R antagonist antibody is administered to the patient at each dosing interval in a single bolus injection, for example, using an injection device as described further below. For example, a fixed dose of 1,575 mg of the IGF-1R antagonist antibody can be administered to a patient as a single bolus injection of 1,575 mg, optionally with a large volume autoinjector or on-body injection device containing the 1,575 mg dose, at each dosing interval (e.g. once every two weeks). In other embodiments, the entire amount of the fixed dose of the IGF-1R antagonist antibody is administered to the patient as two or more consecutive bolus injections. By way of example, a fixed dose of 1,575 mg of the IGF-1R antagonist antibody can be administered to the patient in three consecutive injections of 525 mg each, optionally with three injection devices (e.g. autoinjectors) each containing a 525 mg dose, at each dosing interval (e.g. once every two weeks). Consecutive injections given within the period of a single day are considered to be a single administration within the context of the invention. In other words, by way of example, administration of a fixed dose of 1,575 mg once every two weeks can be given either as a single bolus injection of 1,575 mg administered to the patient once every two weeks or three consecutive bolus injections of 525 mg each administered to the patient within the period of one day once every two weeks.

The pharmaceutical compositions described above can be filled into vials, syringes, autoinjectors, on-body injectors, patch injectors, cartridges, or other containers or delivery devices and optionally packaged with instructions for use (e.g. prescribing information containing instructions for using the pharmaceutical compositions for treating thyroid eye disease) to prepare pharmaceutical products. In certain embodiments, the pharmaceutical compositions described herein are incorporated into an injection device (e.g. a self-administration injection device). Such devices are commercially available and include, but are not limited to, autoinjectors, dosing pens, microinfusion pumps, syringe pumps, patch injectors, on-body injectors, and pre-filled syringes. Exemplary devices in which the pharmaceutical compositions for use according to the methods of the invention can be incorporated include autoinjectors (e.g., SureClick®, EverGentle®, Avanti®, DosePro®, Molly®, Leva®, ConfiPen™, Aerio™, ArQ™-Bios, ARAI™, YpsoMate® 5.5, and Maggie® 5.0), pen injection devices (e.g., Madie® pen injector, DCP™ pen injector, BD Vystra™ disposable pen, and BD™ reusable pen), on-body injectors (e.g. BD Evolve™, BD Libertas™, enFuse®, Sorrel™ on-body delivery system, SmartDose® 10, Vertiva®, and Symbioze®), pre-filled syringes (BD Sterifill™, BD Hypak™, prefilled syringes from Baxter), syringe pumps, and patch injectors (e.g. YpsoDose®). In some embodiments, the IGF-1R antagonist antibody or pharmaceutical compositions comprising the IGF-1R antagonist antibody are administered to the patient with an injection device having an injection volume of at least 1 mL. In other embodiments, the IGF-1R antagonist antibody or pharmaceutical compositions comprising the IGF-1R antagonist antibody are administered to the patient with an injection device having an injection volume of at least 3 mL. In still other embodiments, the IGF-1R antagonist antibody or pharmaceutical compositions comprising the IGF-1R antagonist antibody are administered to the patient with an injection device having an injection volume of at least 5 mL. In yet other embodiments, the IGF-1R antagonist antibody or pharmaceutical compositions comprising the IGF-1R antagonist antibody are administered to the patient with an injection device having an injection volume of at least 10 mL. Suitable high-volume injection devices for administering the IGF-1R antagonist antibody or pharmaceutical compositions comprising the IGF-1R antagonist antibody according to the methods of the invention are described in Schneider et al., Expert Opinion on Drug Delivery, Vol. 20: 815-830, 2023, which is hereby incorporated by reference. In certain embodiments, the IGF-1R antagonist antibody or pharmaceutical compositions comprising the IGF-1R antagonist antibody are administered to the patient with an on-body injection device.

The following examples, including the experiments conducted and the results achieved, are provided for illustrative purposes only and are not to be construed as limiting the scope of the appended claims.

### EXAMPLES

### Example 1. Population Pharmacokinetic Analysis of Teprotumumab

Teprotumumab is approved in the U.S. for the treatment of thyroid eye disease (TED) regardless of disease activity or duration. The recommended dosing for teprotumumab is an initial intravenous infusion of 10 mg/kg followed by seven subsequent intravenous infusions of 20 mg/kg every 3 weeks. Each infusion is administered over 60 to 90 minutes. Development of a subcutaneous, fixed-dose regimen would provide patients with an alternative, less invasive route of administration with shorter administration times for each dose as well as provide a treatment option for patients with venous access issues. The subcutaneous, fixed dosing also reduces the complexity of dose preparation and administration.

A population pharmacokinetic (PK) analysis was performed to quantitatively describe the clinical PK of teprotumumab and identify sources of interindividual variability in active TED patients and healthy subjects following intravenous (IV) and subcutaneous (SC) administration. A nonlinear mixed effects modeling (NONMEM) approach, with the first-order conditional estimation with interaction (FOCEI) method in NONMEM software, version 7.4.3 (ICON, Maryland), was used for the population PK analysis.

### Studies Included in the Analysis

A population PK model for teprotumumab was developed based on data collected from four clinical studies: TED01RV (ClinicalTrials.gov ID NCT01868997), HZNP-TEP-102 (ClinicalTrials.gov ID NCT06563856), HZNP-TEP-103 (ClinicalTrials.gov ID NCT06389578) and HZN-TEP-301 (ClinicalTrials.gov ID NCT03298867). The design and results of the TED01RV and HZN-TEP-301 trials are described in Smith et al., N Engl J. Med., Vol. 376:1748-1761, 2017 and Douglas et al., N Engl J Med., Vol. 382: 341-352, 2020, respectively.

The HZNP-TEP-102 study was a Phase 1, open-label, single center, single ascending dose trial that assessed the safety, tolerability, and PK of teprotumumab administered SC to healthy adult participants. This study had 4 cohorts, including 2 cohorts dosed SC (750 mg or 1,500 mg), 1 cohort dosed IV (1,500 mg), and 1 cohort dosed SC (1,500 mg) with ENHANZE® Drug Product. The lyophilized formulation of teprotumumab, which contained per vial 500 mg of teprotumumab, L-histidine (7.82 mg), L-histidine hydrochloride monohydrate (33.43 mg), polysorbate 20 (1.05 mg), and trehalose dihydrate (993 mg), was used for this study. The lyophilized powder was reconstituted with sterile water for injection and administered SC with a syringe pump or IV depending on the cohort. The trial consisted of a Screening Period (Days −28 to −2), check-in (Day −1), Treatment Day (Day 1), Follow-up Period (Days 11 through 57), and an End-of-study Visit (Day 71). Participants were confined to the clinical unit from Day −1 until discharge on Day 8. PK and safety (including immunogenicity) endpoints were evaluated. Serum teprotumumab exposures (Cₘₐₓ and AUC) increased in an approximately dose-proportional manner from 750 mg to 1,500 mg. Maximum serum concentrations were observed approximately 4 to 5 days post-dose with SC administration. Teprotumumab SC bioavailability was 56.1% for teprotumumab alone at the 1,500 mg dose. Teprotumumab was safe and well tolerated in healthy participants when administered as a single SC infusion dose of 750 or 1,500 mg. There were no deaths, serious adverse events (SAEs), or adverse events (AEs) leading to investigational product discontinuation. Overall, 16 of 37 participants (43.2%) experienced 29 treatment emergent adverse events (TEAEs) during the study, all of which were mild (Grade 1) and resolved by the end of the study. No treatment-related trends were observed. Among the 16 participants who received a single SC infusion of teprotumumab (either 750 mg or 1,500 mg), 3 experienced 5 injection site reactions (ISRs), which included injection site erythema (2 events), injection site hemorrhage, injection site pain and injection site edema. An additional 2 participants experienced menstruation delayed. No events of hyperglycemia, hearing impairment, anaphylaxis or systemic infusion-related reactions occurred during the study. No participant had a positive anti-drug antibody (ADA) result after a single dose of SC teprotumumab. No new safety signals were identified.

The HZNP-TEP-103 is a Phase 1b, open-label, PK, safety and tolerability study evaluating a single SC dose of teprotumumab in two different formulations—a lyophilized formulation and a high concentration formulation—followed by the FDA-approved IV treatment regimen for TEPEZZA® (teprotumumab-trbw) in TED participants. The lyophilized formulation contained per vial 500 mg of teprotumumab, L-histidine (7.82 mg), L-histidine hydrochloride monohydrate (33.43 mg), polysorbate 20 (1.05 mg), and trehalose dihydrate (993 mg). When reconstituted with 10 mL of Sterile Water for Injection, the final formulation concentrations of the components were 50 mg/mL teprotumumab, 20 mM histidine/histidine HCl, 250 mM trehalose, and 0.01% (weight/volume (w/v)) polysorbate 20 at pH 5.5. The high concentration formulation contained 150 mg/mL teprotumumab, 20 mM histidine/histidine HCl, 210 mM trehalose, 40 mM methionine and 0.2% (w/v) poloxamer 188 at pH 5.5. Cohort 1 participants received via a syringe pump a single SC injection of 1,400 mg teprotumumab prepared from the lyophilized formulation followed by 8 teprotumumab IV infusions (10 mg/kg for initial infusion followed by seven 20 mg/kg infusions every 3 weeks) starting at 6 weeks after the SC injection. Cohort 2 participants received via a syringe pump a single SC injection of 1,400 mg teprotumumab in the high concentration formulation followed by 8 teprotumumab IV infusions (10 mg/kg for initial infusion followed by seven 20 mg/kg infusions every 3 weeks) starting at 6 weeks after the SC injection. Cohort 1 enrolled 6 participants, whereas cohort 2 enrolled 10 participants. Participants were followed for 6 weeks during the SC treatment period prior to the first IV injection. This study has completed the PK portion, and the open-label phase is ongoing. A summary of the preliminary PK results from the HZNP-TEP-103 study is shown in Table 1 below. All parameters shown as arithmetic mean (CV %) except tmax, which is shown as median (range).

TABLE 1
Summary of the Preliminary PK results from Study HZNP-TEP-103

| | | Injection SC in | | Injection SC in | | | |
|---|---|---|---|---|---|---|---|
| Formulation | PK Parameter | Abdomen | | Thigh | | Overall | |
| Lyophilized | N | 3 | | 3 | | 6 | |
| | Cₘₐₓ (μg/mL) | 90.7 | (43.9) | 117 | (53.8) | 104 | (47.5) |
| | tₘₐₓ (h) | 97.6 | (67.0-119) | 140 | (68.0-142) | 108 | (67.0-142) |
| | t_{1/2} (h) | 411 | (11.7) | 417 | (4.4) | 413 | (8.6) |
| | AUC_{inf} (h*μg/mL) | 57900 | (44.2) | 71900 | (62.4) | 64900 | (51.7) |
| | AUCₗₐₛₜ (h*μg/mL) | 46000 | (41.0) | 61900 | (67.0) | 53900 | (55.8) |
| High | N | 5 | | 5 | | 10 | |
| Concentration | Cₘₐₓ (μg/mL) | 53.0 | (74.4) | 85.4 | (38.4) | 69.2 | (55.2) |
| | tₘₐₓ (h) | 167 | (97.9-335) | 91.3 | (69.0-94.8) | 96.4 | (69.0-335) |
| | t_{1/2} (h) | 396 | (19.6) | 564 | (43.9) | 489 | (41.3) |
| | AUC_{inf} (h*μg/mL) | 40100 | (78.3) | 55700 | (80.1) | 44600 | (72.6) |
| | AUCₗₐₛₜ (h*μg/mL) | 31700 | (77.4) | 45100 | (43.8) | 38400 | (57.7) |

Based on preliminary PK results, the high concentration formulation exhibited lower mean PK exposure (AUC and Cmax) than the lyophilized formulation and the SC injection on thigh had mean higher exposure than the SC injection on abdomen. A preliminary safety analysis for the SC treatment period includes TEAEs experienced prior to the first IV infusion.

In Cohort 1, 4 of 6 participants (66.7%) experienced 6 TEAEs, of which 2 were mild and 4 were moderate in intensity; no event occurred in more than 1 participant. Two participants reported TEAEs that were considered related to teprotumumab by the Investigator. There were no deaths, SAEs or adverse events leading to investigational product discontinuation, and there was no discernable pattern of TEAEs between injections in the abdomen or thigh. Three participants in Cohort 1 (50.0%) experienced an adverse event of special interest (AESI), including 2 events of hyperglycemia and 1 ISR. One participant with type 2 diabetes experienced blood glucose increased of 1-day duration, and another with screening glycated hemoglobin (HbAlc) of 5.8% experienced Type 2 diabetes and began treatment with metformin. The third participant experienced injection site rash that resolved in 2 days. There were no events of hearing impairment or inflammatory bowel disease during the SC treatment period.

In Cohort 2, 7 of 10 participants (70.0%) experienced 25 TEAEs, of which 24 were mild and 1 was moderate in intensity. The most commonly reported TEAE was muscle spasms (4 participants) followed by diarrhea, headache, nausea and menstrual disorder (heavy menstrual bleeding, oligomenorrhoea), which were experienced by 2 participants each. Six participants reported 18 TEAEs that were considered related to teprotumumab by the Investigator. There were no deaths, SAEs or adverse events leading to investigational product discontinuation, and there was no discernable pattern of TEAEs between injections in the abdomen or thigh. One participant experienced 2 AESIs, injection site pain and injection site itching, which resolved within 2 hours. There were no events of systemic hypersensitivity reactions, hyperglycemia, hearing impairment or inflammatory bowel disease during the SC treatment period. No new safety concerns were identified from clinical laboratory values, vital sign measurements or changes in weight, and none were considered clinically significant or reported as a TEAE by the Investigator in either Cohort 1 or Cohort 2.

SC administration of a high concentration of teprotumumab was generally well tolerated at the local injection site. No events of anaphylaxis, systemic infusion-related reactions, hearing impairment or exacerbation of inflammatory bowel disease at 6 weeks of treatment were observed. No new safety signals were identified in either cohort.

Concentrations of teprotumumab in serum samples were determined using a fully validated immunoassay. Samples for determination of teprotumumab concentrations in serum were analyzed using a validated ELISA in study TED01RV and a validated electrochemiluminescent (ECL) sandwich method in studies HZNP-TEP-102, HZNP-TEP-103, and HZNP-TEP-301 (see Xin et al., Clinical Pharmacokinetics, Vol. 60: 1029-1040, 2021). The lower limit of quantification (LLOQ) in serum is 10 ng/mL for studies HZNP-TEP-102, HZNP-TEP-103, and HZNP-TEP-301. The LLOQ in serum is 76.3 ng/mL for the TED01RV study.

### Population PK Model Development

Based on the known PK property of teprotumumab, the default structural model is a two-compartment model with first-order elimination from the central compartment and redistribution from the peripheral compartment. The population PK model was parameterized in terms of clearance from the central compartment (CL), volume of the central compartment (V_{c}), clearance of distribution from the central to the peripheral compartment (Q), volume of the peripheral compartment (Vₚ), absorption rate constant (ka), and bioavailability after SC administration (F₁). Alternative model structures (such as the 1-compartment model and 3-compartment model) were also explored, as appropriate. The final base model was chosen based on the objective function value, goodness-of-fit plots, and reliability of model parameter estimates. The impact of baseline age, body weight, sex, ethnicity (Hispanic vs. non-Hispanic), race (Asian, Black, White, and Other), health status (healthy volunteer vs. thyroid eye disease patient), smoking status (non-user vs. user), formulation (lyophilized vs. high concentration), and hepatic and renal function-related parameters (e.g. bilirubin, alanine aminotransferase, aspartate aminotransferase and creatinine clearance) on the PK of teprotumumab was investigated. Covariates were selected using a forward addition and backward elimination method (based on the significant levels p<0.01 and p<0.001, respectively). After completion of the model development process, the final population PK model was evaluated with multiple model qualification/validation methods, including diagnostic plots, prediction-corrected visual predictive check (pcVPC), numerical predictive check (NPC), bootstrap, and shrinkage assessments.

The final population PK analysis dataset consisted of 1,118 observed serum concentrations from 125 subjects enrolled in 4 clinical studies of teprotumumab in subjects with active TED or healthy volunteers. A two-compartment model with first-order absorption and elimination best characterized teprotumumab PK following IV or SC administration. The final population PK model well described the teprotumumab PK data following IV and SC administration, as assessed by diagnostic plots, individual fits, pcVPC, and NPC results.

In the final population PK model, baseline body weight and formulation were identified as statistically significant covariates on the PK of teprotumumab. Other covariates, including baseline age, sex, ethnicity, race, bilirubin, alanine aminotransferase, aspartate aminotransferase, creatinine clearance, smoking status, and health status, did not show a statistically significant impact on the PK of teprotumumab. For a typical subject with a body weight of 74 kg, the estimated CL was 0.267 L/day (% relative standard error [RSE]=2.51%), V_{c} was 3.08 L (% RSE =2.44%), Q was 0.527 L/day (% RSE=7.89%) and Vₚ was 3.46 L (% RSE=4.38%). Absorption rate constant (ka) after SC administration was 0.227 day⁻¹ (% RSE=7.40%). A 10% decrease in body weight resulted in 10.4% decreased CL and 7.81% decreased V_{c}. Bioavailability (F₁) values after SC administration were 55.0% (% RSE=12.50%) and 37.8% (% RSE=12.40%) for the lyophilized and high concentration formulations, respectively. Interindividual variability on CL, V_{c}, Q, Vₚ, and ka were 23.9%, ²1.1%, 43.7%, 36.9%, and 44.6%, respectively. A summary of key population PK parameters and covariate effects is presented in Table 2 below. The geometric mean elimination half-life of teprotumumab was 20.7 days with a coefficient of variation (CV) of 22.9% in TED patients.

TABLE 2
Key Population PK Parameters and Covariate Effects

| | | | | Change |
|---|---|---|---|---|
| | | Estimate | | from |
| PK Parameters and Covariates | | (% RSE) | | Typical (%) |
| Typical Clearance, CL (L/day, body weight = 74 kg) | | 0.267 | (2.51%) | — |
| Body weight (kg) | 10^{th} percentile (57.9 kg) | 0.207 | | −22.6 |
| | 90^{th} percentile (101 kg) | 0.369 | | 38.3 |
| Typical Central Volume, V_{c} (L) | | 3.08 | (2.44%) | — |
| Body weight (kg) | 10^{th} percentile (57.9 kg) | 2.55 | | −17.2 |
| | 90^{th} percentile (101 kg) | 3.92 | | 27.1 |
| Typical Inter-Compartmental Clearance, Q (L/day) | | 0.527 | (7.89%) | — |
| Typical Peripheral Volume, Vₚ (L) | | 3.46 | (4.38%) | — |
| Typical Absorption Rate Constant, Ka (1/day) | | 0.227 | (7.40%) | — |
| Bioavailability after | High Concentration | 37.8 | (12.40%) | — |
| SC administration, F₁ | Formulation | | | |
| (%) | Lyophilized Formulation | 55.0 | (12.50%) | 17.6 |

### Population PK Model Simulations for SC Dose Selection

To support the SC dose selection for the high concentration formulation, the developed population PK model was applied to predict teprotumumab exposure. The teprotumumab concentration-time profiles were simulated following the approved dosage using IV lyophilized formulation (10 mg/kg for the first IV infusion and 20 mg/kg every 3 weeks (Q3W) for the remaining 7 IV infusions) as well as proposed SC high concentration formulation regimens.

A total of 1,000 patients were simulated using the final model parameter estimates and estimated subject-specific random effects. The simulation conditions were as follows:
Treatment group
   Lyophilized formulation (reference formulation)
      1st dose 10 mg/kg, followed by 20 mg/kg Q3W, a total of 8 IV infusions
   High Concentration formulation (test formulation)
      1,575 mg SC every 2 weeks (Q2W) for 24 weeks
      1,732.5 mg SC Q2W for 24 weeks (10% increased dose)
IV infusion duration
   1.5 hours for first and second doses, 1 hour for remaining doses
No. of simulated TED patients
   1,000 per treatment group
Body weight
   Sampled from TED patient population (mean=79.6 kg, SD=21.2 kg, range: 47.6-168.7 kg)
Exposure metrics
   Maximum concentration at steady state (Cmax,ss), minimum or trough concentration at steady state (Cmin,ss), and average concentration at steady state (Cavg,ss)

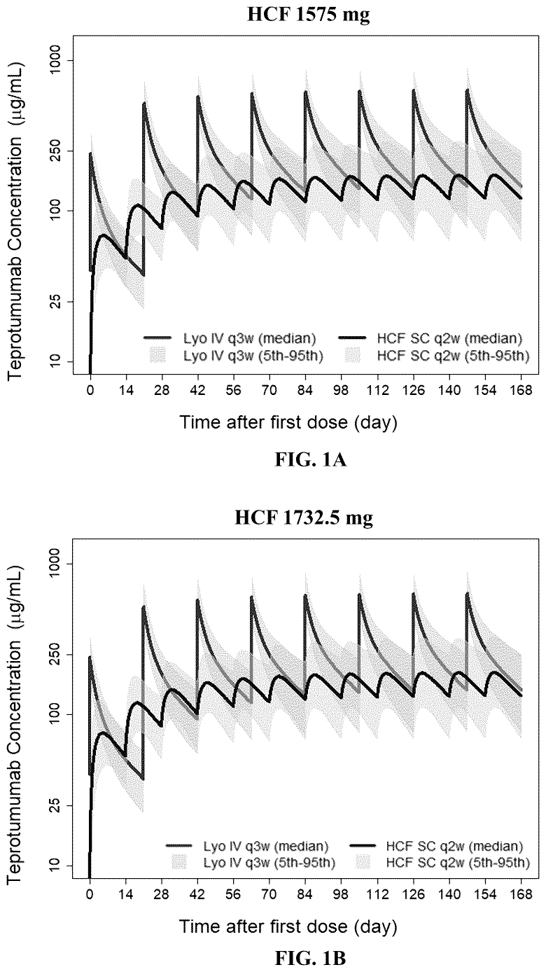

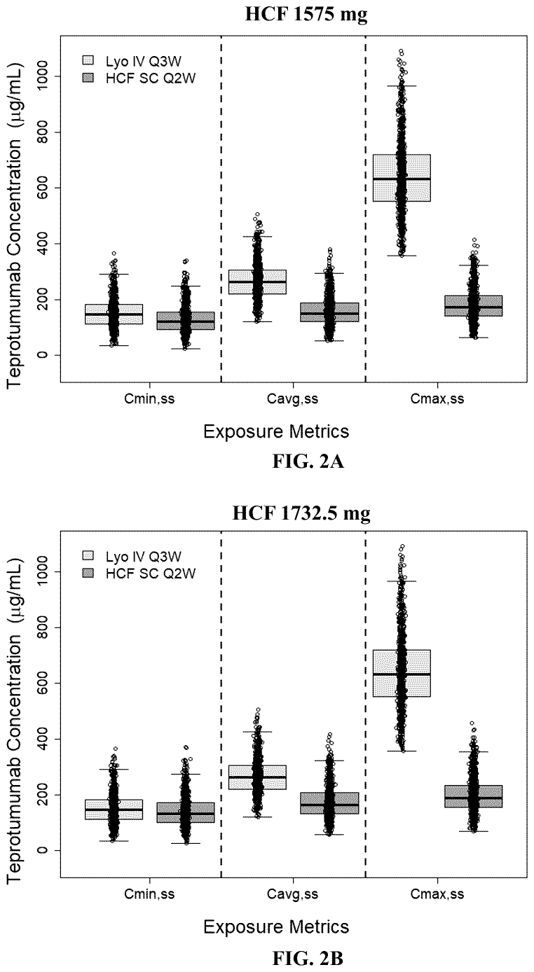

The average, maximum, and minimum concentrations at one dosing interval after the last IV dose at week 21 and the last SC dose at week 22 (Cavg,ss, Cmax,ss, and Cmin,ss) were computed. Patients' Cavg,ss, Cmax,ss, and Cmin,ss values were natural log-transformed and analyzed using a linear mixed effects model with fixed effect terms for formulation. The geometric mean ratio (GMR) under lyophilized (lyo) formulation IV and high concentration formulation (HCF) SC for Cavg,ss, Cmax,ss, and Cmin,ss of teprotumumab (HCF SC Q2W/Lyo IV Q3W) were calculated to demonstrate the non-inferiority of HCF SC Q2W to Lyo IV Q3W. The confidence intervals were the least square means estimate of the difference ±the standard error of the difference in the least square means times a t-value. The lower and upper values were then exponentiated (back-transformed) to give a confidence interval on the ratio of the means. The level of significance to accept the alternative hypothesis was set at 0.05. The criteria for acceptance of the proposed fixed-dose SC regimen were that the SC regimen should achieve similar steady-state Cmin,ss as the approved IV regimen, defined as the GMR of the steady-state Cmin,ss (SC vs. IV) close to 1 and the lower 90% bound of the GMR greater or equal to 0.8. A summary of the PK parameters of teprotumumab following the IV Q3W regimen with the lyophilized formulation or the SC Q2W regimen with the high concentration formulation is shown in Table 3 below. The simulated time-course PK profiles are shown in FIGS. 1A and 1B, whereas a comparison of exposure metrics between treatment groups is shown in FIGS. 2A and 2B.

TABLE 3
Summary of Teprotumumab PK Between SC and Reference IV Formulations

| | Lyo IV Q3W | | HCF SC | | | | |
|---|---|---|---|---|---|---|---|
| | (reference) | | Q2W (test) | | | Test/Reference | |
| | | 95% | Dose | | 95% | | 90% |
| PK Parameters¹ | GM | CI | (mg) | GM | CI | GMR | CI |
| Cmin, ss | 143.5 | (140.3, 146.8) | 1,575 | 120.2 | (117.4, 123.2) | 0.838 | (0.815, 0.861) |
| (μg/mL) | | | 1,732.5 | 132.3 | (129.1, 135.5) | 0.921 | (0.896, 0.947) |
| Cavg, ss | 261.6 | (257.8, 265.5) | 1,575 | 151.4 | (148.2, 154.6) | 0.579 | (0.566, 0.591) |
| (μg/mL) | | | 1,732.5 | 166.5 | (163.0, 170.0) | 0.636 | (0.623, 0.650) |
| Cmax, ss | 633.2 | (625.3, 641.2) | 1,575 | 173.3 | (170.2, 177.2) | 0.274 | (0.269, 0.280) |
| (μg/mL) | | | 1,732.5 | 191.1 | (187.3, 195.0) | 0.302 | (0.296, 0.308) |

¹Back-transformed geometric mean and confidence interval calculated using natural log-transformed values with confidence interval obtained based on the t distribution. GM = geometric mean; GMR = geometric mean ratio; CI = confidence interval

Based on 1000 simulated TED patients per treatment group, the geometric mean (95% CI) values of Cmin,ss were 143.5 (140.3, 146.8) μg/mL, 120.2 (117.4, 123.3) μg/mL, and 132.3 (129.1, 135.5) μg/mL following the approved Lyo IV Q3W dose regimen, the proposed HCF SC dose regimens of 1,575 mg Q2W or 1,732.5 mg Q2W, respectively. The GMR and 90% confidence intervals for Cmin,ss were 0.838 (0.815, 0.861) and 0.921 (0.896, 0.947) for SC doses in the HCF of 1,575 mg and 1,732.5 mg, respectively. One hundred trial simulations showed 95% and 100% trials with a lower limit of GMR>0.8 for a SC dose of 1,575 mg and 1,732.5 mg, respectively. The HCF 1,575 mg SC Q2W regimen was considered the appropriate fixed dose SC regimen by achieving the pre-defined criteria of demonstrating a geometric mean ratio (90%) of 0.838 (0.815, 0.861) with the lower bound of 90% CI above 0.8, compared to the approved Lyo IV Q3W dose regimen. All of the participants achieved a minimum concentration at steady state (Cmin,ss) >20 μg/mL, a target efficacious concentration for >90% IGF-1R occupancy.

To explore the SC dose range, for each dose group, a total of 1,000 patients were simulated using the final model parameter estimates and estimated subject-specific random effects. Simulation results for proposed doses are summarized in Table 4 below. The simulation conditions were as follows:
Treatment group
   Lyophilized formulation (reference formulation)
      1st dose 10 mg/kg, followed by 20 mg/kg Q3W, a total of 8 IV infusions
   High Concentration formulation (test formulation)
      Ranged from 1,575 mg to 2,000 mg SC Q2W for 24 weeks
      Ranged from 725 mg to 1,200 mg SC Q1W for 24 weeks
IV infusion duration
   1.5 hours for first and second doses, 1 hour for remaining doses
No. of simulated TED patients
   1,000 per treatment group
Body weight
   Sampled from TED patient population (mean=79.6 kg, SD=21.2 kg, range: 47.6-168.7 kg)
Exposure metric: Cmin,ss
Proposed target
   SC dose can achieve a geometric mean ratio (GMR)>1 and the lower and upper bounds of 90% CI>1 (target 1)
   SC dose can achieve the lower bound of 90% CI>0.8 and the upper bound of 90% CI>1 (target 2)

TABLE 4
Simulated SC Dosing Regimens of Teprotumumab
in High Concentration Formulation

| Every Other Week (Q2W) | | | | Weekly (Q1W) | | | |
|---|---|---|---|---|---|---|---|
| Dosing Interval | | | | Dosing Interval | | | |
| SC dose | | Lower | Upper | SC dose | | Lower | Upper |
| (mg) | GMR | CI | CI | (mg) | GMR | CI | CI |
| 1,575 | 0.838 | 0.815 | 0.861 | 775 | 0.960 | 0.935 | 0.986 |
| 1,600 | 0.851 | 0.828 | 0.875 | 800 | 0.991 | 0.965 | 1.107 |
| 1,625 | 0.864 | 0.841 | 0.889 | 825 | 1.022 | 0.995 | 1.049 |
| 1,650 | 0.878 | 0.854 | 0.902 | 850 | 1.053 | 1.025 | 1.081 |
| 1,675 | 0.891 | 0.867 | 0.916 | 875 | 1.084 | 1.056 | 1.113 |
| 1,700 | 0.904 | 0.879 | 0.930 | 900 | 1.115 | 1.086 | 1.144 |
| 1,725 | 0.917 | 0.892 | 0.943 | 925 | 1.146 | 1.116 | 1.176 |
| 1,750 | 0.931 | 0.905 | 0.957 | 950 | 1.177 | 1.146 | 1.208 |
| 1,775 | 0.944 | 0.918 | 0.971 | 975 | 1.208 | 1.176 | 1.240 |
| 1,800 | 0.957 | 0.931 | 0.984 | 1,000 | 1.239 | 1.206 | 1.272 |
| 1,825 | 0.971 | 0.944 | 0.998 | 1,025 | 1.270 | 1.237 | 1.303 |
| 1,850 | 0.984 | 0.957 | 1.012 | 1,050 | 1.301 | 1.267 | 1.335 |
| 1,875 | 0.997 | 0.970 | 1.025 | 1,075 | 1.332 | 1.297 | 1.367 |
| 1,900 | 1.011 | 0.983 | 1.039 | 1,100 | 1.362 | 1.327 | 1.399 |
| 1,925 | 1.024 | 0.996 | 1.053 | 1,125 | 1.393 | 1.357 | 1.431 |
| 1,950 | 1.037 | 1.009 | 1.066 | 1,150 | 1.424 | 1.387 | 1.462 |
| 1,975 | 1.050 | 1.022 | 1.080 | 1,175 | 1.455 | 1.418 | 1.494 |
| 2,000 | 1.064 | 1.035 | 1.094 | 1,200 | 1.486 | 1.448 | 1.526 |

For the Q2W dosing interval, doses of 1,850 mg and above met the target 2 criteria, whereas doses of 1,950 mg and above met the target 1 criteria. For the Q1W dosing interval, doses of 800 mg and above met the target 2 criteria, whereas doses of 850 mg and above met the target 1 criteria.

The relationship between the model-predicted steady-state Cmin,ss and the week 24 proptosis responses was analyzed with linear logistic regression models based on 99 TED patients. Proptosis response was defined as ≥2 mm reduction from baseline level of proptosis in the study eye without deterioration (e.g. ≥2 mm increase) of proptosis in the fellow eye. Table 5 summarizes the model-predicted Cmin,ss and probability of proptosis response for each Cmin,ss decile for the HCF SC dosing regimen (1,575 mg Q2W) and the IV dosing regimen with the lyophilized formulation (10 mg/kg for initial infusion followed by 20 mg/kg infusions Q3W).

TABLE 5
Relationship Between Minimum Steady-State Teprotumumab Concentrations and Proptosis Response

| | HCF SC Dosing¹ | | | Lyo IV Dosing² | | | | | |
|---|---|---|---|---|---|---|---|---|---|
| | Model- | | | Model- | | | Lyo IV Dosing² | | |
| | predicted | Probability | | predicted | Probability | | Observed | Probability | |
| Cmin, ss | Cmin, ss | of proptosis | | Cmin, ss | of proptosis | | Cmin, ss | of proptosis | |
| deciles | (range, μg/mL) | Median | 90% CI | (range, μg/mL) | Median | 90% CI | (range, μg/mL) | Median | 90% CI |
| Q1 | (44.46, | 0.371 | (0.124, | (46.20, | 0.394 | (0.142, | (76.10, | 0.632 | (0.463, |
| | 62.65) | | 0.707) | 66.68) | | 0.714) | 95.90) | | 0.772) |
| Q2 | (64.67, | 0.517 | (0.281, | (68.96, | 0.551 | (0.329, | (99.32, | 0.684 | (0.550, |
| | 82.86) | | 0.739) | 89.44) | | 0.747) | 110.0) | | 0.792) |
| Q3 | (84.88, | 0.627 | (0.452, | (91.72, | 0.660 | (0.514, | (115.0, | 0.722 | (0.614, |
| | 103.1) | | 0.771) | 112.2) | | 0.782) | 122.0) | | 0.809) |
| Q4 | (105.1, | 0.706 | (0.587, | (114.5, | 0.742 | (0.638, | (123.0, | 0.743 | (0.639, |
| | 123.3) | | 0.802) | 135.0) | | 0.820) | 127.0) | | 0.820) |
| Q5 | (125.3, | 0.767 | (0.675, | (137.2, | 0.795 | (0.710, | (128.7, | 0.756 | (0.657, |
| | 143.5) | | 0.836) | 157.7) | | 0.862) | 150.4) | | 0.829) |
| Q6 | (145.5, | 0.810 | (0.721, | (160.0, | 0.836 | (0.746, | (151.0, | 0.810 | (0.721, |
| | 163.7) | | 0.874) | 180.5) | | 0.900) | 158.0) | | 0.874) |
| Q7 | (165.7, | 0.843 | (0.751, | (182.8, | 0.866 | (0.769, | (165.0, | 0.831 | (0.741, |
| | 183.9) | | 0.907) | 203.2) | | 0.927) | 172.8) | | 0.895) |
| Q8 | (185.9, | 0.868 | (0.771, | (205.5, | 0.888 | (0.785, | (176.0, | 0.856 | (0.760, |
| | 204.1) | | 0.929) | 226.0) | | 0.947) | 196.0) | | 0.918) |
| Q9 | (206.1, | 0.887 | (0.785, | (228.3, | 0.904 | (0.797, | (202.0, | 0.886 | (0.785, |
| | 224.3) | | 0.947) | 248.8) | | 0.961) | 216.0) | | 0.946) |
| Q10 | (226.3, | 0.902 | (0.795, | (251.0, | 0.918 | (0.807, | (222.7, | 0.898 | (0.792, |
| | 244.5) | | 0.959) | 271.5) | | 0.971) | 268.0) | | 0.956) |

¹HCF SC dosing: 1,575 mg SC Q2W for 24 weeks
²Lyo IV dosing: 1^{st} dose 10 mg/kg, followed by 20 mg/kg Q3W, a total of 8 IV infusions

As shown in Table 5, a fixed dose of 1,575 mg administered SC once every 2 weeks (Q2W) in the high concentration formulation is predicted to achieve a model-predicted Cmin,ss and probability of proptosis response for each Cmin,ss decile similar to the IV formulation at the approved clinical IV dosing (1st dose 10 mg/kg, followed by 20 mg/kg doses Q3W for a total of 8 IV infusions).

In summary, teprotumumab PK following intravenous or subcutaneous administration can be adequately described by a two-compartment linear disposition model with first-order absorption and elimination. Baseline body weight and formulation were identified as statistically significant covariates on the PK of teprotumumab. However, other covariates, including baseline age, sex, ethnicity, race, smoking status, health status (healthy subjects vs. TED patients), and liver and renal function tests (e.g. bilirubin, alanine aminotransferase, aspartate aminotransferase, and creatinine clearance), did not show statistically significant impact on the PK of teprotumumab. Based on simulation results, a fixed dose of 1,575 mg Q2W in the high concentration SC formulation (150 mg/mL teprotumumab) was predicted to achieve the dose-selection criteria. Specifically, this dosing regimen was predicted to have a steady-state Cmin concentration of 120.2 μg/mL, which is expected to maintain >90% target saturation systemically similar to the approved IV dosing regimen throughout the dosing interval by having a geometric mean ratio (SC vs. IV) of Cmin,ss of 0.838 with 90% CI of 0.815 to 0.861 with the lower bound CI above the pre-specified noninferiority margin of 0.8. The steady-state Cmin concentration achieved with the proposed SC dosing regimen is predicted to have a comparable probability of proptosis response as the IV dosing regimen and therefore, is expected to be efficacious. The proposed total of 12 doses is the same treatment duration as the approved IV dosing regimen.

The safety profile of the selected SC dose of 1,575 mg and Q2W dosing interval is expected to not be significantly different from that of the IV dosing regimen because the SC regimen is predicted to achieve similar steady-state Cmin,ss as the approved IV dose regimen for teprtoumumab, but with lower maximum serum concentration (Cmax) and area under the curve (AUC) during the 24-week treatment duration. No new safety signals have been identified with SC administration in the completed and ongoing Phase 1 studies of teprotumumab SC compared with the experience to date noted with approved IV dosing regimen.

### Example 2. a Phase 3, Randomized, Double-Masked, Placebo-Controlled, Parallel-Group, Multicenter Trial to Evaluate the Efficacy, Safety and Tolerability of Subcutaneous Teprotumumab in Participants with Moderate-to-Severe Active Thyroid Eye Disease

The overall objective of this phase 3 study was to evaluate the efficacy, safety, and tolerability of teprotumumab administered as a fixed dose subcutaneously (SC) in a high concentration formulation in comparison to placebo in the treatment of patients with moderate to severe active TED. Approximately 80 adult participants with active TED who met the eligibility criteria were randomized in a 1:1 ratio to receive 12 injections of teprotumumab or placebo every 2 weeks (Q2W). All participants entered a 24-week double-masked treatment period, during which teprotumumab or placebo was administered SC on Day 1 (Baseline) and weeks 2, 4, 6, 8, 10, 12, 14, 16, 18, 20, and 22 with a final double-masked treatment period visit at Week 24. The primary endpoint was the effect of teprotumumab versus placebo on the proptosis responder rate at Week 24. Proptosis responder rate was assessed as the percentage of participants with a ≥2 mm reduction from Baseline in proptosis in the study eye without deterioration (i.e. ≥2 mm increase) of proptosis in the fellow eye).

After providing written informed consent, participants entered the screening phase (up to 42 days) during which eligibility of the participants was assessed. Participants were eligible for the study if they met all of the following inclusion criteria:
Male or female between the ages of 18 and 80 years, inclusive, at Screening.
A clinical diagnosis of Graves' disease associated with active TED with a clinical activity score (CAS)≥3 (on the 7-item scale) for the most severely affected eye at Screening and Baseline (Day 1).
Moderate-to-severe active TED (not sight-threatening but has an appreciable impact on daily life), usually associated with 1 or more of the following:
   Lid retraction ≥2 mm,
   Moderate or severe soft tissue involvement, and/or
   Inconstant or constant diplopia
Proptosis ≥3 mm from Baseline (prior to diagnosis of TED), as estimated by treating physician, and/or proptosis ≥3 mm above normal for race and gender.
Onset of active TED symptoms (as determined by participant records) within 15 months prior to Baseline.
Euthyroid with the baseline disease under control or have mild hypo- or hyperthyroidism (defined as free thyroxine (FT4) and free triiodothyronine (FT3) levels <50% above or below the normal limits) at Screening. Every effort was made to correct the mild hypo- or hyperthyroidism promptly and to maintain the euthyroid state for the full duration of the trial.
Did not require immediate surgical ophthalmological intervention and was not planning corrective surgery/irradiation during the trial.
Women of childbearing potential (including those with an onset of menopause <2 years prior to Screening, non-therapy-induced amenorrhea for <12 months prior to Screening or not surgically sterile [absence of ovaries and/or uterus]) must have had a negative serum pregnancy test at Screening and negative urine pregnancy tests at all protocol-specified time points (ie, prior to each dose and throughout participation in the trial).
Women of childbearing potential who were sexually active with a non-vasectomized male partner must have agreed to use 2 reliable forms of contraception during the trial, 1 of which was recommended to be hormonal, such as an oral contraceptive.
Was willing and able to comply with the protocol requirements for the duration of the trial.

Participants were ineligible for the study if they met any of the following exclusion criteria:
Decreased best-corrected visual acuity due to optic neuropathy, defined by a decrease in vision of 2 lines on the Snellen chart (or equivalent), new visual field defect or color defect secondary to optic nerve involvement within the last 6 months.
Corneal decompensation unresponsive to medical management.
Decrease in CAS of 2 or more points between Screening and Baseline.
Decrease in proptosis of ≥2 mm between Screening and Baseline.
Prior orbital irradiation, orbital decompression or strabismus surgery (excluding childhood strabismus surgeries unrelated to TED/Graves' disease).
Planned to have eyelid surgery during the trial.
Received periocular botulinum toxin injection within 12 months prior to Screening.
Any systemic use of a steroid (IV or oral) or steroid eye drops for the treatment of TED or other conditions within 3 weeks prior to Screening. Exceptions included local administration (excluding periocular), e.g., topical, intra-articular, and inhaled steroids, as well as steroids used to treat infusion reactions.
Used selenium within 3 weeks prior to Screening (selenium must not be restarted during the trial); however, taking a multivitamin that included selenium (<100 mcg daily) was allowed.
Any previous treatment with rituximab (Rituxan® or MabThera®) within 12 months prior to the first administration of investigational product (IP), tocilizumab (Actemra® or Roactemra®) within 6 months prior to the first administration of IP or any non-steroid immunosuppressive agent within 3 months prior to the first administration of IP.
Screening laboratory samples that showed any of the following abnormal results:
   Alanine aminotransferase or aspartate aminotransferase ≥3×the upper limit of normal;
   Estimated glomerular filtration rate ≤30 mL/min/1.73 m².
An HbA1c≥7.5 at Screening.
Pre-existing ophthalmic disease that, in the judgment of the Investigator, would preclude trial participation or complicate interpretation of trial results.
Any history of malignancy unless it had been curatively treated and had not recurred for at least 5 years prior to Screening.
Inflammatory Bowel Disease (ulcerative colitis or Crohn's disease) with active inflammatory intestinal or extra-intestinal symptoms, started or had a change in medical therapy within 3 months prior to Screening, had bowel surgery within 6 months prior to Screening or planned to have bowel surgery during the trial.
Positive for hepatitis B surface antigen (HBsAg).
Negative for HBsAg and positive for hepatitis B core antibody (HBcAb)
Detected sensitivity on hepatitis C virus (HCV) RNA quantitative test for positive HCV antibody or positive human immunodeficiency virus (HIV)-1/HIV-2 antibody
Female and pregnant or breastfeeding (lactating).
Current drug or alcohol abuse, or history of either, within the previous 12 months.
Known hypersensitivity to teprotumumab, its excipients, medical adhesives, or prior hypersensitivity reactions to monoclonal antibodies.
Previously received teprotumumab or previously participated in a teprotumumab clinical trial.
Previously received any anti-insulin like growth factor receptor monoclonal antibody.
Previously received any investigational drug within 60 days or 5 half-lives, whichever was longer, prior to Screening.
Any other condition that, in the opinion of the Investigator, would have made the participant unsuitable for inclusion in the trial.

A participant who did not meet all the inclusion criteria or met any of the exclusion criteria was considered a screen failure. A screen failure was allowed to rescreen once for the trial if both the Investigator and Sponsor agreed regarding rescreening.

Adult participants with active TED who met the trial eligibility criteria were randomized on Day 1 (Baseline) in a 1:1 ratio to receive 12 SC injections of 1,575 mg of teprotumumab or placebo every 2 weeks (Q2W). Up to 25% of participants with no diplopia at Baseline could be enrolled. The randomization was stratified by presence of diplopia at baseline (yes vs. no), tobacco use status (yes vs. no) and region (Japan vs. non-Japan). All enrolled participants entered a 24 week Double-masked Treatment Period, during which teprotumumab or placebo was administered SC on Day 1 (Baseline) and Weeks 2, 4, 6, 8, 10, 12, 14, 16, 18, 20, and 22. A final Double-masked Treatment Period visit occurred at Week 24.

At the end of the Double-masked Treatment Period (Week 24), all participants were assessed for treatment response: proptosis responders (study eye had ≥2-mm reduction in proptosis from Baseline without deterioration (i.e. ≥2-mm increase in proptosis) in fellow eye) or proptosis non-responders (i.e. study eye had <2-mm reduction in proptosis). Proptosis non-responders who completed the Double-masked Treatment Period could choose to enter the Open-label Treatment Period and receive 12 SC injections of open-label teprotumumab (1,575 mg dose) Q2W at Weeks 24, 26, 28, 30, 32, 34, 36, 38, 40, 42, 44 and 46. Participants returned to the clinic at Week 48 for end-of-treatment assessments and was contacted by phone/email 30 days after the Week 48 Visit for safety assessment. Proptosis responders, as well as non-responders who chose not to enter the Open-label Treatment Period, entered a Follow-up Period, during which investigational product was not administered. These participants were contacted by phone/email 30 days after the Week 24 Visit for safety assessment.

At each injection, teprotumumab was administered at a fixed dose of 1,575 mg in approximately 10.5 mL via an on-body injection (OBI) device. The on-body device (SmartDose® 10 On-Body Delivery System, West Pharmaceutical Services, Inc.) is a single-use, battery-powered, wearable device with a separate, pre-fillable, polymer-based cartridge that is filled with the drug product. For the treatment group, each cartridge contained a drug product formulation comprising 150 mg/mL teprotumumab, 20 mM histidine/histidine HCl, 210 mM trehalose, 40 mM methionine, and 0.2% (w/v) poloxamer 188, at pH 5.5. For the placebo group, each cartridge contained a similar formulation without teprotumumab. For each injection, the Investigator or trained designee loaded the pre-filled cartridge (drug product or placebo) into the device and applied the device to the participant. After activation, the device is configured to automatically insert an injection needle into the participant's SC tissue (thigh or abdomen) and deliver the investigational product (drug product or placebo) at an injection rate of approximately 1 mL/min until the cartridge content delivery is complete (approximately 12 minutes, which includes device activation time). Upon dose completion, the injector was removed and returned to the clinical site for disposal.

As described in Example 1, a fixed dose of 1,575 mg teprotumumab administered once every 2 weeks is predicted from population PK modeling, including preliminary data from a phase 1 SC trial of teprotumumab with the same high concentration formulation, to achieve similar steady-state minimum concentrations (Cmin,ss) of teprotumumab as the approved IV regimen. The predicted Cmin,ss for this dosing regimen is also associated with a similar probability of proptosis response as the approved IV regimen (see Example 1) and therefore is expected to be efficacious. This dosing regimen is expected to maintain systemic exposures throughout the dosing interval above 20 μg/mL, the target trough concentration associated with 90% target inhibition for IV teprotumumab (see Xin et al., Clinical Pharmacokinetics, Vol. 60: 1029-1040, 2021).

### Trial Endpoints and Assessments

The primary endpoint for the study was to evaluate the effect of teprotumumab versus placebo on the proptosis responder rate at Week 24. Proptosis responder rate was assessed as the percentage of participants with a 2 mm or greater reduction from Baseline (Day 1) in proptosis in the study eye without deterioration (i.e. 2 mm or greater increase) of proptosis in the fellow eye.

Secondary endpoints to further evaluate the effect of teprotumumab versus placebo on other measures of clinical efficacy and patient-reported outcomes included the following:
Mean change from Baseline at Week 24 in proptosis measurement in the study eye
Overall responder rate (percentage of participants with ≥2-point reduction in CAS AND ≥2-mm reduction in proptosis from Baseline, provided there is no corresponding deterioration (i.e. ≥2-point/mm increase) in CAS or proptosis in the fellow eye) at Week 24
Percentage of participants with a CAS value of 0 or 1 at Week 24 in the study eye
Change from Baseline at Week 24 in diplopia as ordinal response categories
Diplopia responder rate, defined as the percentage of participants with Baseline binocular diplopia >0 who have a reduction of ≥1 grade at Week 24
Complete diplopia responder rate, defined as the percentage of participants with a Baseline binocular diplopia score >0 and a score of 0 at Week 24
Mean change from Baseline at Week 24 in the GO-QoL questionnaire (see Terwee et al., Br. J. Ophthalmol. Vol. 82(7):773-779, 1998) overall score

Exploratory endpoints included additional measures of clinical efficacy, such as motility component of the Clinical Measures of Severity, study eye intraocular pressure, tear production, and change in serum biomarkers. The PK profile of teprotumumab following SC administration was also measured. To assess the safety and tolerability of teprotumumab SC administration, incidence of treatment emergent adverse events (TEAEs), serious adverse events (SAEs), adverse events of special interest (AESIs) (e.g. injection site reactions, infusion-related reactions, hyperglycemia, hearing impairment, new onset or exacerbation of inflammatory bowel disease), adverse device effects (ADEs) and serious adverse device effects (SADEs) were determined. Incidence of anti-drug antibodies and ototoxicity were also measured.

Assessments performed only at Screening and/or Baseline (Day 1) included review of eligibility criteria; collection of demographics including iris pigmentation, medical/surgical history, and prior medications data; and measurement of height. The medical history included documentation of pre-existing hearing loss. Efficacy was assessed by proptosis (measured as proptosis evaluation of the Clinical Measures of Severity using a Hertel exophthalmometer instrument provided by the Sponsor for consistency in measurement), diplopia (measured as part of the Clinical Measures of Severity), CAS (7-item scale), quality of life (using the GO-QoL questionnaire), motility restriction, intraocular pressure (measured by the Goldmann applanation tonometer) and eye tear production (measured by the Schirmer test).

Proptosis was measured for each eye at Screening, Day 1 (Baseline), Weeks 6, 12, 18 and 24 (or premature withdrawal (PW1)) and Weeks 30, 36, 42 and 48 (or premature withdrawal (PW2)) for proptosis non-responders who elected to receive open-label teprotumumab only. A decrease of 2 mm or greater in proptosis from Baseline was considered a response. Diplopia was assessed at Screening, Day 1 (Baseline), Weeks 6, 12, 18 and 24 (or PW1) and Weeks 30, 36, 42 and 48 (or PW2) for proptosis non-responders who elected to receive open-label teprotumumab only. Subjective diplopia scores were as follows:
0=no diplopia;
1=intermittent (diplopia in primary position of gaze, when tired or when first awakening);
2=inconstant (diplopia at extremes of gaze);
3=constant (continuous diplopia in primary or reading position)
   A Baseline score >0 and decrease of ≥1 grade was considered a response. Motility was assessed at Screening, Day 1 (Baseline), Weeks 6, 12, 18 and 24 (or PW1) and Weeks 30, 36, 42 and 48 (or PW2) for proptosis non-responders who elected to receive open-label teprotumumab only by estimating the degrees of restriction in eye movements. Monocular excursions in horizontal and vertical directions of gaze were recorded using the light reflex test (Dolman et al., Ophthalmology, Vol. 119(2):382-389 2012). An increase ≥8° in at least 1 direction of gaze was considered a response.

The CAS assessment (Table 6) was completed at Screening, Day 1 (Baseline), Weeks 6, 12, 18 and 24 (or PW1) and Weeks 30, 36, 42 and 48 (or PW2) for proptosis non-responders who elected to receive open-label teprotumumab only using the 7-item European Group on Graves' Ophthalmopathy amended CAS (Mourits et al., Br. J. Ophthalmol., Vol. 73(8): 639-644, 1989).

TABLE 6
Clinical Activity Score Assessment

| Item¹ | Description |
|---|---|
| 1 | Spontaneous orbital pain |
| 2 | Gaze evoked orbital pain |
| 3 | Eyelid swelling that is considered to be |
| | due to active (inflammatory phase) |
| | thyroid eye disease/Graves' ophthalmopathy |
| 4 | Eyelid erythema |
| 5 | Conjunctival redness that is considered to |
| | be due to active (inflammatory phase) |
| | thyroid eye disease/Graves' ophthalmopathy |
| | (ignore “equivocal” redness) |
| 6 | Chemosis |
| 7 | Inflammation of caruncle or plica |

¹Each item is scored (1 = present; 0 = absent) and scores for each item are summed for a total score.

The GO-QoL questionnaire (Terwee et al., Br. J. Ophthalmol. Vol. 82(7):773-779, 1998) was completed at Day 1 (Baseline), Weeks 6, 12 and 24 (or PW1) and Weeks 30, 36 and 48 (or PW2) for proptosis non-responders who elected to receive open-label teprotumumab only. The GO-QoL is a 16-item self-administered questionnaire divided into 2 subscales that is used to measure changes over time in visual functioning and appearance of a TED patient. Intraocular pressure was measured at Day 1 (Baseline) and Weeks 6, 12, 18 and 24 (or PW1) using the Goldmann applanation tonometer. If possible, the same Goldmann tonometer was used consistently for a participant throughout the trial. Eye tear production was measured at Day 1 (Baseline) and Week 24 (or PW1) by the Schirmer test (Brott and Ronquillo, In: StatPearls [Internet]. Treasure Island (FL): StatPearls Publishing; 2021).

Efficacy analyses will be performed on the Full Analysis Set (FAS), consisting of participants who were randomized to investigational product (either teprotumumab or placebo) and received at least 1 dose of investigational product. The Per Protocol Analysis Set will include participants who were randomized to investigational product (either teprotumumab or placebo), received at least 1 dose of investigational product, completed the Double-masked Treatment Period and did not incur any major protocol violations that would challenge the validity of their data. Safety analyses will be performed on the Safety Analysis Set, consisting of participants who received at least 1 dose of investigational product. The PK Analysis Set will include all participants who received at least 1 dose of investigational product and had at least 1 post-dose PK sample.

The primary analyses will be conducted using the FAS. The primary efficacy endpoint will be analyzed using the Cochran-Mantel-Haenszel (CMH) test adjusted for the randomization stratification factors (presence of diplopia at Baseline, tobacco use status and region). The 4 secondary categorical efficacy endpoints (overall responder rate, CAS (value of 0 or 1, diplopia responder and complete diplopia responder) will be analyzed using the same method as described for the primary efficacy endpoint. To control the overall Type 1 error rate of the trial, the secondary efficacy endpoints will be tested sequentially only if statistical significance is achieved for the primary efficacy endpoint at the significance level of 0.05. For each secondary efficacy endpoint, teprotumumab will be tested against placebo at the 0.05 level only if teprotumumab is statistically significantly different from placebo for the efficacy endpoint preceding it in a hierarchical order. All participants with at least 1 measurable teprotumumab serum concentration post teprotumumab treatment will be included in the summaries of PK. Serum concentrations of teprotumumab will be summarized by visit.

All participants who receive at least 1 dose of investigational product will be included in the safety summaries. The number and percentage of participants experiencing at least 1 TEAE, SAE, ADE, SADE, TEAE resulting in premature discontinuation of investigational product and AESI (injection site reaction, infusion-related reaction, hyperglycemia, hearing impairment, new onset inflammatory bowel disease and exacerbation of inflammatory bowel disease) for each unique System Organ Class and Preferred Term will be summarized by treatment group. TEAEs and SAEs will also be summarized by severity and relationship to investigational product, as assessed by the Investigator. Grade 3 and higher TEAEs, as graded by the National Cancer Institute Common Terminology Criteria for Adverse Events (NCI-CTCAE), will also be summarized for each unique System Organ Class and Preferred Term. All participants who receive at least 1 dose of teprotumumab and have at least 1 sample obtained post teprotumumab treatment will be included in the immunogenicity summary. Incidence rate and titer of positive ADAs will be summarized by visit.

The primary analysis is planned after all participants complete the Follow-up Period (up to 30 days) following the Week 24 Visit in the Double-masked Treatment Period or withdraw from the trial prior to Week 24. Data collected up to the database lock for the primary Week 24 analysis, including some AEs reported during the Open-label Treatment Period, will be included. The follow-up analysis is planned after all participants complete the Follow-up Period (up to 30 days) following the Week 48 Visit or terminate early from the trial.

The phase 3 study results are expected to show that a greater proportion of TED patients receiving teprotumumab at a fixed dose of 1,575 mg Q2W for 24 weeks will be proptosis responders (i.e. have a ≥2 mm reduction from Baseline in proptosis in the study eye) at Week 24 as compared to the proportion of TED patients receiving placebo. The proptosis responder rate in the teprotumumab-treated patients is anticipated to be between 65% and 80% as compared to a proptosis responder rate between 10% and 25% in the placebo-treated patients. It is anticipated that approximately 73% of trial participants will have diplopia at Baseline and of these participants, about 50% to 65% treated with teprotumumab will have a complete diplopia response (i.e. no diplopia at Week 24) as compared to about 25% to 30% of participants receiving placebo. Based on the population PK modeling described in Example 1, the anticipated mean change in proptosis from Baseline in TED patients receiving teprotumumab according to the SC dosing regimen is about −2.80 mm to about −3.30 mm compared to an anticipated mean change in proptosis from Baseline of about 0 mm to about −0.50 mm in TED patients receiving placebo. Because the fixed dose 1,575 mg Q2W regimen is predicted to achieve similar steady-state minimum teprotumumab concentrations (Cmin,ss) as the approved IV regimen (see Example 1), the efficacy and safety profile is expected to be comparable or even improved as compared to the profile when teprotumumab is administered according to the approved IV regimen. Without being bound by theory, the SC dosing regimen may result in fewer adverse events because the SC dosing regimen is predicted to produce lower maximum serum concentrations (Cmax) and area under the curve (AUC) during the 24-week treatment duration. See FIG. 1A, 2A, and Table 3 in Example 1.

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain using no more than routine experimentation, many equivalents to the specific embodiments of the invention described herein. Such equivalents are intended to be encompassed by the following claims.

TABLE 7
Sequence Listing

| SEQ | | |
|---|---|---|
| ID | | |
| NO: | Description | Amino Acid Sequence |
| 1 | Anti-IGF-1R | SYGMH |
| | CDRH1 | |
| 2 | Anti-IGF-1R | IIWFDGSSTYYADSVRG |
| | CDRH2 | |
| 3 | Anti-IGF-1R | ELGRRYFDL |
| | CDRH3 | |
| 4 | Anti-IGF-1R | RASQSVSSYLA |
| | CDRL1 | |
| 5 | Anti-IGF-1R | DASKRAT |
| | CDRL2 | |
| 6 | Anti-IGF-1R | QQRSKWPPWT |
| | CDRL3 | |
| 7 | Anti-IGF-1R | QVELVESGGGVVQPGRSQRLSCAASGFTFSSYGMHWVRQAPG |
| | VH | KGLEWVAIIWFDGSSTYYADSVRGRFTISRDNSKNTLYLQMN |
| | | SLRAEDTAVYFCARELGRRYFDLWGRGTLVSVSS |
| 8 | Anti-IGF-1R | EIVLTQSPATLSLSPGERATLSCRASQSVSSYLAWYQQKPGQ |
| | VL | APRLLIYDASKRATGIPARFSGSGSGTDFTLTISSLEPEDFA |
| | | VYYCQQRSKWPPWTFGQGTKVESK |
| 9 | Anti-IGF-1R | QVELVESGGGVVQPGRSQRLSCAASGFTFSSYGMHWVRQAPG |
| | Heavy Chain | KGLEWVAIIWFDGSSTYYADSVRGRFTISRDNSKNTLYLQMNS |
| | | LRAEDTAVYFCARELGRRYFDLWGRGTLVSVSSASTKGPSVFP |
| | | LAPSSKSTSGGTAALGCLVKDYFPEPVTVSWNSGALTSGVHTF |
| | | PAVLQSSGLYSLSSVVTVPSSSLGTQTYICNVNHKPSNTKVDK |
| | | KVEPKSCDKTHTCPPCPAPELLGGPSVFLFPPKPKDTLMISRT |
| | | PEVTCVVVDVSHEDPEVKFNWYVDGVEVHNAKTKPREEQYNST |
| | | YRVVSVLTVLHQDWLNGKEYKCKVSNKALPAPIEKTISKAKGQ |
| | | PREPQVYTLPPSRDELTKNQVSLTCLVKGFYPSDIAVEWESNG |
| | | QPENNYKTTPPVLDSDGSFFLYSKLTVDKSRWQQGNVFSCSVM |
| | | HEALHNHYTQKSLSLSPGK |
| 10 | Anti-IGF-1R | EIVLTQSPATLSLSPGERATLSCRASQSVSSYLAWYQQKPGQAP |
| | Light Chain | RLLIYDASKRATGIPARFSGSGSGTDFTLTISSLEPEDFAVYYC |
| | | QQRSKWPPWTFGQGTKVESKRTVAAPSVFIFPPSDEQLKSGTAS |
| | | VVCLLNNFYPREAKVQWKVDNALQSGNSQESVTEQDSKDSTYSL |
| | | SSTLTLSKADYEKHKVYACEVTHQGLSSPVTKSFNRGEC |
| 11 | Anti-IGF-1R | pEVELVESGGGVVQPGRSQRLSCAASGFTFSSYGMHWVRQAPG |
| | Heavy Chain | KGLEWVAIIWFDGSSTYYADSVRGRFTISRDNSKNTLYLQMNS |
| | with post- | LRAEDTAVYFCARELGRRYFDLWGRGTLVSVSSASTKGPSVFP |
| | translational | LAPSSKSTSGGTAALGCLVKDYFPEPVTVSWNSGALTSGVHTF |
| | modifications | PAVLQSSGLYSLSSVVTVPSSSLGTQTYICNVNHKPSNTKVDK |
| | | KVEPKSCDKTHTCPPCPAPELLGGPSVFLFPPKPKDTLMISRT |
| | | PEVTCVVVDVSHEDPEVKFNWYVDGVEVHNAKTKPREEQYNST |
| | | YRVVSVLTVLHQDWLNGKEYKCKVSNKALPAPIEKTISKAKGQ |
| | | PREPQVYTLPPSRDELTKNQVSLTCLVKGFYPSDIAVEWESNG |
| | | QPENNYKTTPPVLDSDGSFFLYSKLTVDKSRWQQGNVFSCSVM |
| | | HEALHNHYTQKSLSLSPG |

1. A method of treating thyroid eye disease in a patient in need thereof comprising administering subcutaneously to the patient an insulin-like growth factor-1 receptor (IGF-1R) antagonist antibody at a dose from about 1,575 mg to about 1,750 mg once every two weeks, wherein the IGF-1R antagonist antibody comprises (i) a heavy chain variable region comprising a CDRH1 having the sequence of SEQ ID NO: 1, a CDRH2 having the sequence of SEQ ID NO: 2, and a CDRH3 having the sequence of SEQ ID NO: 3, and (ii) a light chain variable region comprising a CDRL1 having the sequence of SEQ ID NO: 4, a CDRL2 having the sequence of SEQ ID NO: 5, and a CDRL3 having the sequence of SEQ ID NO: 6.

2. The method of claim 1, wherein the dose is about 1,575 mg.

3. The method of claim 1, wherein the dose is about 1,750 mg.

4. The method of claim 1, wherein the IGF-1R antagonist antibody is administered to the patient in a pharmaceutical composition comprising the antagonist antibody at a concentration from about 150 mg/mL to about 200 mg/mL.

5. The method of claim 4, wherein the pharmaceutical composition further comprises a buffer, a stabilizer, and a surfactant.

6. The method of claim 5, wherein the buffer is a histidine buffer.

7. The method of claim 5, wherein the stabilizer is trehalose.

8. The method of claim 5, wherein the surfactant is polysorbate 20.

9. The method of claim 5, wherein the surfactant is poloxamer 188.

10. The method of claim 4, wherein the pharmaceutical composition further comprises methionine.

11. The method of claim 4, wherein the pharmaceutical composition comprises about 150 mg/mL to about 200 mg/mL of the IGF-1R antagonist antibody, about 15 mM to about 25 mM histidine, about 200 mM to about 275 mM trehalose, and about 0.005% (w/v) to about 0.05% (w/v) polysorbate 20, wherein the composition has a pH of about 5.0 to about 6.0.

12. The method of claim 11, wherein the pharmaceutical composition comprises about 150 mg/mL of the IGF-1R antagonist antibody, about 20 mM histidine, about 250 mM trehalose, and about 0.01% (w/v) polysorbate 20, wherein the composition has a pH of about 5.5.

13. The method of claim 4, wherein the pharmaceutical composition comprises about 150 mg/mL to about 200 mg/mL of the IGF-1R antagonist antibody, about 15 mM to about 25 mM histidine, about 200 mM to about 275 mM trehalose, about 30 mM to about 50 mM methionine, and about 0.05% (w/v) to about 0.35% (w/v) poloxamer 188, wherein the composition has a pH of about 5.0 to about 6.0.

14. The method of claim 13, wherein the pharmaceutical composition comprises about 150 mg/mL of the IGF-1R antagonist antibody, about 20 mM histidine, about 210 mM trehalose, about 40 mM methionine, and about 0.2% (w/v) poloxamer 188, wherein the composition has a pH of about 5.5.

15. The method of claim 4, wherein the pharmaceutical composition is administered to the patient with an injection device pre-filled with the pharmaceutical composition.

16. The method of claim 1, wherein the IGF-1R antagonist antibody is a human monoclonal antibody.

17. The method of claim 16, wherein the antibody is an IgG1 antibody.

18. The method of claim 1, wherein the IGF-1R antagonist antibody comprises a heavy chain variable region comprising the sequence of SEQ ID NO: 7, and a light chain variable region comprising the sequence of SEQ ID NO: 8.

19. The method of claim 1, wherein the IGF-1R antagonist antibody comprises a heavy chain comprising the sequence of SEQ ID NO: 9, and a light chain comprising the sequence of SEQ ID NO: 10.

20. The method of claim 1, wherein the IGF-1R antagonist antibody is teprotumumab.

21. The method of claim 1, wherein the patient is diagnosed with active thyroid eye disease.

22. The method of claim 1, wherein the patient is diagnosed with inactive thyroid eye disease.

23. The method of claim 1, wherein the patient is diagnosed with moderate to severe thyroid eye disease.

24. The method of claim 1, wherein the patient has inconstant or constant diplopia prior to administration of the IGF-1R antagonist antibody.

25. The method of claim 1, wherein the patient has an increase in proptosis of 3 mm or more in at least one eye prior to administration of the IGF-1R antagonist antibody.

26. The method of claim 1, wherein the patient is administered the IGF-1R antagonist antibody over the course of 24 weeks.

27. The method of claim 26, wherein proptosis is reduced by at least 2 mm in at least one eye of the patient following administration of the IGF-1R antagonist antibody.

28. (canceled)

29. The method of claim 26, wherein the severity of diplopia in the patient is reduced following administration of the IGF-1R antagonist antibody.

30. The method of claim 26, wherein the clinical activity score (CAS) of the patient is reduced by at least two points following administration of the IGF-1R antagonist antibody.

31. The method of claim 30, wherein the patient has a CAS of 0 or 1 following administration of the IGF-1R antagonist antibody.

32. The method of claim 26, wherein extraocular muscle volume in at least one eye of the patient is reduced by at least 25% following administration of the IGF-1R antagonist antibody.

33. The method of claim 26, wherein orbital fat volume in at least one eye of the patient is reduced by at least 30% following administration of the IGF-1R antagonist antibody.

34-37. (canceled)

38. The method of claim 1, wherein the patient has not previously had orbital irradiation, orbital decompression surgery, or strabismus surgery.

39-120. (canceled)
